(19) United States
(12) Patent Application Publication
(10) Pub. No.: US 2022/0168360 A1
(43) Pub. Date: Jun. 2, 2022

(54) COMPOSITIONS, DEVICES, AND METHODS FOR TREATING RESPIRATORY DISORDERS

(71) Applicant: BREATHE RESTORE, INC., Long Lake, MN (US)

(72) Inventors: Gary M. PETRUCCI, Long Lake, MN (US); Jihad A. Mustapha, Ada, MI (US)

(21) Appl. No.: 17/539,595

(22) Filed: Dec. 1, 2021

Related U.S. Application Data
(60) Provisional application No. 63/120,625, filed on Dec. 2, 2020.

(51) Int. Cl. A61K 35/50

(52) U.S. Cl. CPC A61K 35/50; A61M 11/006

(57) ABSTRACT

Compositions, devices, and methods for treating one or more respiratory disorders in a subject (e.g., a mammal) are provided. In an exemplary embodiment, a breathing system is provided and include a pressure-assisted breathing device, and a composition comprising amniotic fluid, an amnion tissue preparation, or a combination thereof. The pressure-assisted breathing device can be a mechanical ventilator. The breathing system can further include a delivery device selected from a nebulizer, a metered dose inhaler, or a dry powder inhaler operably connected to the breathing system to deliver the composition into the breathing system.

### CROSS REFERENCE TO RELATED APPLICATIONS

This application claims the benefit under 35 U.S.C. § 119(e) of U.S. provisional application No. 63/120,625, filed on Dec. 2, 2020, the disclosure of which is herein incorporated by reference in its entirety.

### TECHNICAL FIELD

This document relates to compositions, devices, and methods for treating and preventing respiratory disorders or conditions in a subject (e.g., a human). For example, the compositions, devices, and methods provided herein can be used in treating subjects having a respiratory or non-respiratory disorder to deliver therapeutic agents into a subject's respiratory tract.

### BACKGROUND

Respiratory disorders can be treated through inhalation of various therapeutic compositions. Typically, therapeutic compositions are inhaled as aerosols. Various devices can provide aerosols to a subject's lungs, such as inhalers, nebulizers, or mechanical ventilators.

### SUMMARY

This document provides compositions comprising amniotic fluid, an amnion tissue preparation, or a combination thereof. This document also provides various breathing systems and devices, including ventilators, nebulizers, inhalers, vaping devices, and e-cigarettes, comprising an inhalable composition comprising amniotic fluid or an amnion tissue preparation. Methods for using compositions, breathing systems, and devices are also provided herein. For example, this document provides methods for using compositions (e.g., inhalable formulations) and devices described herein for treating or preventing respiratory disorders.

In some embodiments, methods provided herein can provide long-term maintenance treatment following an acute treatment of a respiratory disorder (including but not limited to acute respiratory disorders). In some embodiments, methods provided herein can regenerate or restore respiratory tissue (including but not limited to lung tissue) or respiratory function. In some embodiments, methods provided herein can include treatment of subjects requiring mechanical breathing assistance (e.g. mechanical ventilation), spontaneously breathing subjects with artificial airways, or ambulatory subjects capable of independent, spontaneous breathing.

In some embodiments, methods provided herein can treat or prevent respiratory disorders and conditions, including bronchospasms, COPD, chronic bronchitis, asthma, emphysema, pulmonary hypertension, interstitial lung disease, pulmonary fibrosis, pneumonia, interstitial pneumonia, lung infections, idiopathic pulmonary fibrosis, covid-19, coronavirus, acute respiratory distress syndrome, and infections such SARS-CoV-2, SARS-CoV, MERS, and Pertussis.

In one aspect, an inhalable composition is provided, comprising amniotic fluid. In some embodiments, the composition can optionally further comprise stem cells, a stem cell preparation, or combinations thereof. In some embodiments, the composition can optionally further comprise one or more active agents selected from acetyl cysteine, aclidinium bromide, albuterol, albuterol sulfate, amikacin sulfate, an amnion tissue preparation, arformoterol sulfate, atropine sulfate, aztreonam, beclomethasone dipropionate, bitolterol mesylate, budesonide, ciclesonide, cromolyn sodium, desflurane, dexamethasone sodium phosphate, dornase alfa, enflurane, epinephrine, ergotamine tartrate, flunisolide, fluticasone propionate, fomoterol fumarate, glycopyrrolate, halothane, indacaterol maleate, iloprost, insulin, ipratropium bromide, isoetharine hydrochloride, isoflurane, isoproterenol hydrochloride, levalbuterol hydrochloride, levodopa, loxapine, mannitol, metaproterenol sulfate, methacholine chloride, mometasone furoate, nedocromil sodium, nicotine, nitric oxide, olodaterol hydrochloride, pentamidine isethionate, pentetate calcium trisodium, pentetate zinc trisodium, pirbuterol acetate, revefenacin, ribavirin, salmeterol xinafoate, sevoflurane, terbutaline sulfate, tetrahydrocannabinol, cannabidiol, tiotropium bromide, tobramycin, trimcinolone acetonide, umeclidinium bromide, vilanterol trifenatate, xenon xe-133, zanamivir, epinephrine, sodium chloride, interferon beta, interferon beta 1-b, interferon beta gene delivery, interferon beta-1a, a BKB2R antagonist, a KLKB1 inhibitor, androgens, recombinant SERPING1, vitamin D, a HAS2 or HAS3 inhibitor, timbetasin, and combinations thereof.

In another aspect, a breathing system is provided, comprising a pressure-assisted breathing device; and a composition comprising amniotic fluid, an amnion tissue preparation, or a combination thereof. In some embodiments, the breathing system can optionally include one or more of the following features. The pressure-assisted breathing device can be a mechanical ventilator. The pressure-assisted breathing device can be selected from the group consisting of an intensive care ventilator, a bubble ventilator, a continuous positive airway pressure system, a bi-level positive airway pressure system, an automatic positive airway pressure system, and an adaptive servo ventilation system. The breathing system can further comprise a delivery device selected from a nebulizer, a metered dose inhaler, or a dry powder inhaler. The delivery device can be operably connected to the breathing system to deliver the composition into the breathing system. The amniotic fluid or the amnion tissue preparation can lack viable cells. The amniotic fluid or the amnion tissue preparation can comprise viable cells. The composition can consist essentially of amniotic fluid, an amnion tissue preparation, or a combination thereof. The composition can further comprise stem cells, a stem cell preparation, or combinations thereof. The composition can further comprise one or more active agents selected from acetyl cysteine, aclidinium bromide, albuterol, albuterol sulfate, amikacin sulfate, an amnion tissue preparation, arformoterol sulfate, atropine sulfate, aztreonam, beclomethasone dipropionate, bitolterol mesylate, budesonide, ciclesonide, cromolyn sodium, desflurane, dexamethasone sodium phosphate, dornase alfa, enflurane, epinephrine, ergotamine tartrate, flunisolide, fluticasone propionate, fomoterol fumarate, glycopyrrolate, halothane, indacaterol maleate, iloprost, insulin, ipratropium bromide, isoetharine hydrochloride, isoflurane, isoproterenol hydrochloride, levalbuterol hydrochloride, levodopa, loxapine, mannitol, metaproterenol sulfate, methacholine chloride, mometasone furoate, nedocromil sodium, nicotine, nitric oxide, olodaterol hydrochloride, pentamidine isethionate, pentetate calcium trisodium, pentetate zinc trisodium, pirbuterol acetate, revefenacin, ribavirin, salmeterol xinafoate, sevoflurane, terbutaline sulfate, tetrahydrocannabinol, cannabidiol, tiotropium bromide, tobramycin, trimcinolone acetonide, umeclidinium bromide, vilanterol trifenatate, xenon xe-133, zanamivir, epinephrine, sodium chloride, interferon beta, interferon beta 1-b, interferon beta gene delivery, interferon beta-1a, a BKB2R antagonist, a KLKB1 inhibitor, androgens, recombinant SERPING1, vitamin D, a HAS2 or HAS3 inhibitor, timbetasin, and combinations thereof.

In another aspect, a method is provided for treating a subject having a respiratory disorder or providing prophylaxis to a subject to prevent or reduce the severity of a developing respiratory disorder, the method comprising mechanically ventilating the subject with a breathing system as described herein (for example, as described above).

In another aspect, a method is provided for treating a subject having a respiratory disorder or providing prophylaxis to a subject to prevent or reduce the severity of a developing respiratory disorder, the method comprising mechanically ventilating the subject with a breathing system; and delivering a composition to the subject through the breathing system, wherein the composition comprises amniotic fluid, an amnion tissue preparation, or a combination thereof. In some embodiments, the method can optionally include one or more of the following features. The breathing system can comprise a pressure-assisted breathing device. The pressure-assisted breathing device can be a mechanical ventilator. The pressure-assisted breathing device can be selected from the group consisting of an intensive care ventilator, a bubble ventilator, a continuous positive airway pressure system, a bi-level positive airway pressure system, an automatic positive airway pressure system, and an adaptive servo ventilation system. The breathing system can further comprise a delivery device selected from a nebulizer, a metered dose inhaler, or a dry powder inhaler. The delivery device can be operably connected to the breathing system to deliver the composition into the breathing system. The method can further comprise actuating the delivery device to deliver one or more doses of the composition into the breathing system and into the subject. The composition can be delivered to the subject as a liquid, a solution aerosol, a suspension aerosol, or a nebulized aerosol. The composition can be in the form of an aerosol or vapor in the breathing system. The composition can be delivered in a particulate or droplet form having an average diameter of from about 0.1 microns to about 5 microns. The composition can be delivered in particulate or droplet form having an average diameter of from about 1 micron to about 5 microns. The composition can be delivered in particulate or droplet form having an average diameter of from about 2.5 microns to about 4.5 microns. The composition can be delivered in particulate or droplet form having an average diameter of from about 3.5 microns to about 5 microns. The method can further comprise identifying the subject as having or at risk of developing the respiratory disorder. The method can further comprise identifying the respiratory disorder or one or more symptoms of the respiratory disorder. The respiratory disorder can be selected from chronic obstructive pulmonary disease, asthma, acute asthma, chronic asthma, severe asthma, allergic asthma, bronchial asthma, intrinsic asthma, respiratory distress syndrome of the newborn, reversible respiratory disease, cystic fibrosis, bronchospasms, bronchitis, chronic bronchitis, bronchiectasis, alpha-1 antitrypsin emphysema, emphysema, associated cor pulmonale with pulmonary hypertension, right ventricular hypertrophy and right heart failure, pulmonary hypertension, interstitial lung disease, pulmonary fibrosis, pneumonia, interstitial pneumonia, a lung infection, idiopathic pulmonary fibrosis, cystic fibrosis, tuberculosis, severe acute respiratory syndrome, infection, pulmonary embolus, pulmonary arterial hypertension, pulmonary edema, pneumocystis pneumonia, SARS-CoV-2 infection, covid-19, coronavirus, acute respiratory distress syndrome, intensive care unit (ICU) syndrome, systemic inflammatory response syndrome (SIRS), sepsis, severe sepsis, septic shock, or multiple organ dysfunction syndrome (MODS), cystic fibrosis, sarcoidosis, and combinations thereof, and wherein the non-respiratory disorder is selected from an autoimmune disease, a spondyloarthropathy, an intestinal disease, diabetes, a skin disease, a non-respiratory infection, a pain disorder, intensive care unit (ICU) syndrome, systemic inflammatory response syndrome (SIRS), sepsis, severe sepsis, septic shock, or multiple organ dysfunction syndrome (MODS), cystic fibrosis, sarcoidosis, and combinations thereof. The amniotic fluid or the amnion tissue preparation can lack viable cells. The amniotic fluid or the amnion tissue preparation can comprise viable cells. The composition can consist essentially of amniotic fluid, an amnion tissue preparation, or a combination thereof. The composition can further comprise stem cells, a stem cell preparation, or combinations thereof. The composition can further comprise one or more active agents selected from acetyl cysteine, aclidinium bromide, albuterol, albuterol sulfate, amikacin sulfate, an amnion tissue preparation, arformoterol sulfate, atropine sulfate, aztreonam, beclomethasone dipropionate, bitolterol mesylate, budesonide, ciclesonide, cromolyn sodium, desflurane, dexamethasone sodium phosphate, dornase alfa, enflurane, epinephrine, ergotamine tartrate, flunisolide, fluticasone propionate, fomoterol fumarate, glycopyrrolate, halothane, indacaterol maleate, iloprost, insulin, ipratropium bromide, isoetharine hydrochloride, isoflurane, isoproterenol hydrochloride, levalbuterol hydrochloride, levodopa, loxapine, mannitol, metaproterenol sulfate, methacholine chloride, mometasone furoate, nedocromil sodium, nicotine, nitric oxide, olodaterol hydrochloride, pentamidine isethionate, pentetate calcium trisodium, pentetate zinc trisodium, pirbuterol acetate, revefenacin, ribavirin, salmeterol xinafoate, sevoflurane, terbutaline sulfate, tetrahydrocannabinol, cannabidiol, tiotropium bromide, tobramycin, trimcinolone acetonide, umeclidinium bromide, vilanterol trifenatate, xenon xe-133, zanamivir, epinephrine, sodium chloride, interferon beta, interferon beta 1-b, interferon beta gene delivery, interferon beta-1a, a BKB2R antagonist, a KLKB1 inhibitor, androgens, recombinant SERPING1, vitamin D, a HAS2 or HAS3 inhibitor, timbetasin, and combinations thereof.

In another aspect, a nebulizer is provided, comprising a composition comprising amniotic fluid, an amnion tissue preparation, or a combination thereof. In some embodiments, the nebulizer can optionally include one or more of the following features. The nebulizer can be selected from a jet nebulizer, a soft mist nebulizer, an ultrasonic nebulizer, and a vibrating mesh nebulizer. The amniotic fluid or the amnion tissue preparation can lack viable cells. The amniotic fluid or the amnion tissue preparation can comprise viable cells. The composition can consist essentially of amniotic fluid, an amnion tissue preparation, or a combination thereof. The composition can further comprise stem cells, a stem cell preparation, or combinations thereof. The composition can further comprise one or more active agents selected from acetyl cysteine, aclidinium bromide, albuterol, albuterol sulfate, amikacin sulfate, an amnion tissue preparation, arformoterol sulfate, atropine sulfate, aztreonam, beclomethasone dipropionate, bitolterol mesylate, budesonide, ciclesonide, cromolyn sodium, desflurane, dexamethasone sodium phosphate, dornase alfa, enflurane, epinephrine, ergotamine tartrate, flunisolide, fluticasone propionate, fomoterol fumarate, glycopyrrolate, halothane, indacaterol maleate, iloprost, insulin, ipratropium bromide, isoetharine hydrochloride, isoflurane, isoproterenol hydrochloride, levalbuterol hydrochloride, levodopa, loxapine, mannitol, metaproterenol sulfate, methacholine chloride, mometasone furoate, nedocromil sodium, nicotine, nitric oxide, olodaterol hydrochloride, pentamidine isethionate, pentetate calcium trisodium, pentetate zinc trisodium, pirbuterol acetate, revefenacin, ribavirin, salmeterol xinafoate, sevoflurane, terbutaline sulfate, tetrahydrocannabinol, cannabidiol, tiotropium bromide, tobramycin, trimcinolone acetonide, umeclidinium bromide, vilanterol trifenatate, xenon xe-133, zanamivir, epinephrine, sodium chloride, interferon beta, interferon beta 1-b, interferon beta gene delivery, interferon beta-1a, a BKB2R antagonist, a KLKB1 inhibitor, androgens, recombinant SERPING1, vitamin D, a HAS2 or HAS3 inhibitor, timbetasin, and combinations thereof.

In another aspect, a method is provided for treating a subject having a respiratory disorder, the method comprising administering, by way of ambulatory inhalation from a nebulizer, a composition comprising amniotic fluid, an amnion tissue preparation, or a combination thereof. In some embodiments, the method can optionally include one or more of the following features. The nebulizer can be selected from a jet nebulizer, a soft mist nebulizer, an ultrasonic nebulizer, and a vibrating mesh nebulizer. The composition can be administered in a particulate or droplet form having an average diameter of from about 0.1 microns to about 5 microns. The composition can be administered in particulate or droplet form having an average diameter of from about 1 micron to about 5 microns. The composition can be administered in particulate or droplet form having an average diameter of from about 2.5 microns to about 4.5 microns. The composition can be administered in particulate or droplet form having an average diameter of from about 3.5 microns to about 5 microns. The method can further comprise identifying the subject as having or at risk of developing the respiratory disorder. The method can further comprise identifying the respiratory disorder or one or more symptoms of the respiratory disorder. The respiratory disorder can be selected from chronic obstructive pulmonary disease, asthma, acute asthma, chronic asthma, severe asthma, allergic asthma, bronchial asthma, intrinsic asthma, respiratory distress syndrome of the newborn, reversible respiratory disease, cystic fibrosis, bronchospasms, bronchitis, chronic bronchitis, bronchiectasis, alpha-1 antitrypsin emphysema, emphysema, associated cor pulmonale with pulmonary hypertension, right ventricular hypertrophy and right heart failure, pulmonary hypertension, interstitial lung disease, pulmonary fibrosis, pneumonia, interstitial pneumonia, a lung infection, idiopathic pulmonary fibrosis, cystic fibrosis, tuberculosis, severe acute respiratory syndrome, infection, pulmonary embolus, pulmonary arterial hypertension, pulmonary edema, pneumocystis pneumonia, SARS-CoV-2 infection, covid-19, acute respiratory distress syndrome, intensive care unit (ICU) syndrome, systemic inflammatory response syndrome (SIRS), sepsis, severe sepsis, septic shock, or multiple organ dysfunction syndrome (MODS), cystic fibrosis, sarcoidosis, and combinations thereof, and wherein the non-respiratory disorder is selected from an autoimmune disease, a spondyloarthropathy, an intestinal disease, diabetes, a skin disease, a non-respiratory infection, a pain disorder, intensive care unit (ICU) syndrome, systemic inflammatory response syndrome (SIRS), sepsis, severe sepsis, septic shock, or multiple organ dysfunction syndrome (MODS), cystic fibrosis, sarcoidosis, and combinations thereof. The amniotic fluid or the amnion tissue preparation can lack viable cells. The amniotic fluid or the amnion tissue preparation can comprise viable cells. The composition can consist essentially of amniotic fluid, an amnion tissue preparation, or a combination thereof. The composition can further comprise stem cells, a stem cell preparation, or combinations thereof. The composition can further comprise one or more active agents selected from acetyl cysteine, aclidinium bromide, albuterol, albuterol sulfate, amikacin sulfate, an amnion tissue preparation, arformoterol sulfate, atropine sulfate, aztreonam, beclomethasone dipropionate, bitolterol mesylate, budesonide, ciclesonide, cromolyn sodium, desflurane, dexamethasone sodium phosphate, dornase alfa, enflurane, epinephrine, ergotamine tartrate, flunisolide, fluticasone propionate, fomoterol fumarate, glycopyrrolate, halothane, indacaterol maleate, iloprost, insulin, ipratropium bromide, isoetharine hydrochloride, isoflurane, isoproterenol hydrochloride, levalbuterol hydrochloride, levodopa, loxapine, mannitol, metaproterenol sulfate, methacholine chloride, mometasone furoate, nedocromil sodium, nicotine, nitric oxide, olodaterol hydrochloride, pentamidine isethionate, pentetate calcium trisodium, pentetate zinc trisodium, pirbuterol acetate, revefenacin, ribavirin, salmeterol xinafoate, sevoflurane, terbutaline sulfate, tetrahydrocannabinol, cannabidiol, tiotropium bromide, tobramycin, trimcinolone acetonide, umeclidinium bromide, vilanterol trifenatate, xenon xe-133, zanamivir, epinephrine, sodium chloride, interferon beta, interferon beta 1-b, interferon beta gene delivery, interferon beta-1a, a BKB2R antagonist, a KLKB1 inhibitor, androgens, recombinant SERPING1, vitamin D, a HAS2 or HAS3 inhibitor, timbetasin, and combinations thereof.

In another aspect, a method is provided for treating a subject having a respiratory disorder, the method comprising administering, to lung tissue of the subject, a composition comprising amniotic fluid, an amnion tissue preparation, or a combination thereof, wherein the administering occurs through ambulatory inhalation of the composition by the subject from a delivery device selected from a nebulizer, a metered dose inhaler, and a dry powder inhaler. In some embodiments, the method can optionally include one or more of the following features. The administering can occur after acute treatment of a respiratory disorder. The acute treatment can comprise mechanical ventilation, ambulatory oxygen administration, or a combination thereof. The administering can occur after the subject has been discharged from hospital care, downgraded from intensive care, downgraded from acute care, downgraded from critical care, or removed from acute care treatment. The administering can occur more than 1 day, more than 2 days, more than 3 days, more than 1 week, more than 2 weeks, more than 3 weeks, more than 6 weeks, more than 8 weeks, more than 10 weeks, or more than 15 weeks after the subject has been discharged from hospital care, downgraded from intensive care, downgraded from acute care, downgraded from critical care, or removed from acute care treatment. The administering can include administering once daily, multiple times daily, every other day, weekly, or monthly for a period of from about 1 day to about 10 years following the acute treatment. The respiratory disorder can be selected from chronic obstructive pulmonary disease, asthma, acute asthma, chronic asthma, severe asthma, allergic asthma, bronchial asthma, intrinsic asthma, respiratory distress syndrome of the newborn, reversible respiratory disease, cystic fibrosis, bronchospasms, bronchitis, chronic bronchitis, bronchiectasis, alpha-1 antitrypsin emphysema, emphysema, associated cor pulmonale with pulmonary hypertension, right ventricular hypertrophy and right heart failure, pulmonary hypertension, interstitial lung disease, pulmonary fibrosis, pneumonia, interstitial pneumonia, a lung infection, idiopathic pulmonary fibrosis, cystic fibrosis, tuberculosis, severe acute respiratory syndrome, infection, pulmonary embolus, pulmonary arterial hypertension, pulmonary edema, pneumocystis pneumonia, SARS-CoV-2 infection, covid-19, coronavirus, acute respiratory distress syndrome, intensive care unit (ICU) syndrome, systemic inflammatory response syndrome (SIRS), sepsis, severe sepsis, septic shock, or multiple organ dysfunction syndrome (MODS), cystic fibrosis, sarcoidosis, and combinations thereof, and wherein the non-respiratory disorder is selected from an autoimmune disease, a spondyloarthropathy, an intestinal disease, diabetes, a skin disease, a non-respiratory infection, a pain disorder, intensive care unit (ICU) syndrome, systemic inflammatory response syndrome (SIRS), sepsis, severe sepsis, septic shock, or multiple organ dysfunction syndrome (MODS), cystic fibrosis, sarcoidosis, and combinations thereof. The composition can further comprise stem cells, a stem cell preparation, or combinations thereof. The composition can further comprise one or more active agents selected from acetyl cysteine, aclidinium bromide, albuterol, albuterol sulfate, amikacin sulfate, an amnion tissue preparation, arformoterol sulfate, atropine sulfate, aztreonam, beclomethasone dipropionate, bitolterol mesylate, budesonide, ciclesonide, cromolyn sodium, desflurane, dexamethasone sodium phosphate, dornase alfa, enflurane, epinephrine, ergotamine tartrate, flunisolide, fluticasone propionate, fomoterol fumarate, glycopyrrolate, halothane, indacaterol maleate, iloprost, insulin, ipratropium bromide, isoetharine hydrochloride, isoflurane, isoproterenol hydrochloride, levalbuterol hydrochloride, levodopa, loxapine, mannitol, metaproterenol sulfate, methacholine chloride, mometasone furoate, nedocromil sodium, nicotine, nitric oxide, olodaterol hydrochloride, pentamidine isethionate, pentetate calcium trisodium, pentetate zinc trisodium, pirbuterol acetate, revefenacin, ribavirin, salmeterol xinafoate, sevoflurane, terbutaline sulfate, tetrahydrocannabinol, cannabidiol, tiotropium bromide, tobramycin, trimcinolone acetonide, umeclidinium bromide, vilanterol trifenatate, xenon xe-133, zanamivir, epinephrine, sodium chloride, interferon beta, interferon beta 1-b, interferon beta gene delivery, interferon beta-1a, a BKB2R antagonist, a KLKB1 inhibitor, androgens, recombinant SERPING1, vitamin D, a HAS2 or HAS3 inhibitor, timbetasin, and combinations thereof.

In another aspect, a method is provided for providing maintenance treatment to a subject following an acute treatment of a respiratory disorder in the subject, the method comprising administering, to lung tissue of the subject, after completion of acute treatment of the subject's respiratory disorder, a composition comprising amniotic fluid, an amnion tissue preparation, or a combination thereof. In some embodiments, the method can optionally include one or more of the following features. The acute treatment can comprise mechanical ventilation, ambulatory oxygen administration, or a combination thereof. The administering can occur after the subject has been discharged from hospital care, downgraded from intensive care, downgraded from acute care, downgraded from critical care, or removed from acute care treatment. The administering can occur more than 1 day, more than 2 days, more than 3 days, more than 1 week, more than 2 weeks, more than 3 weeks, more than 6 weeks, more than 8 weeks, more than 10 weeks, or more than 15 weeks after the subject has been discharged from hospital care, downgraded from intensive care, downgraded from acute care, downgraded from critical care, or removed from acute care treatment. The administering can include administering once daily, multiple times daily, every other day, weekly, or monthly for a period of from about 1 day to about 10 years following the acute treatment. The respiratory disorder can be selected from chronic obstructive pulmonary disease, asthma, acute asthma, chronic asthma, severe asthma, allergic asthma, bronchial asthma, intrinsic asthma, respiratory distress syndrome of the newborn, reversible respiratory disease, cystic fibrosis, bronchospasms, bronchitis, chronic bronchitis, bronchiectasis, alpha-1 antitrypsin emphysema, emphysema, associated cor pulmonale with pulmonary hypertension, right ventricular hypertrophy and right heart failure, pulmonary hypertension, interstitial lung disease, pulmonary fibrosis, pneumonia, interstitial pneumonia, a lung infection, idiopathic pulmonary fibrosis, cystic fibrosis, tuberculosis, severe acute respiratory syndrome, infection, pulmonary embolus, pulmonary arterial hypertension, pulmonary edema, pneumocystis pneumonia, SARS-CoV-2 infection, covid-19, acute respiratory distress syndrome, intensive care unit (ICU) syndrome, systemic inflammatory response syndrome (SIRS), sepsis, severe sepsis, septic shock, or multiple organ dysfunction syndrome (MODS), cystic fibrosis, sarcoidosis, and combinations thereof, and wherein the non-respiratory disorder is selected from an autoimmune disease, a spondyloarthropathy, an intestinal disease, diabetes, a skin disease, a non-respiratory infection, a pain disorder, intensive care unit (ICU) syndrome, systemic inflammatory response syndrome (SIRS), sepsis, severe sepsis, septic shock, or multiple organ dysfunction syndrome (MODS), cystic fibrosis, sarcoidosis, and combinations thereof. The composition can further comprise stem cells, a stem cell preparation, or combinations thereof. The composition can further comprise one or more active agents selected from acetyl cysteine, aclidinium bromide, albuterol, albuterol sulfate, amikacin sulfate, an amnion tissue preparation, arformoterol sulfate, atropine sulfate, aztreonam, beclomethasone dipropionate, bitolterol mesylate, budesonide, ciclesonide, cromolyn sodium, desflurane, dexamethasone sodium phosphate, dornase alfa, enflurane, epinephrine, ergotamine tartrate, flunisolide, fluticasone propionate, fomoterol fumarate, glycopyrrolate, halothane, indacaterol maleate, iloprost, insulin, ipratropium bromide, isoetharine hydrochloride, isoflurane, isoproterenol hydrochloride, levalbuterol hydrochloride, levodopa, loxapine, mannitol, metaproterenol sulfate, methacholine chloride, mometasone furoate, nedocromil sodium, nicotine, nitric oxide, olodaterol hydrochloride, pentamidine isethionate, pentetate calcium trisodium, pentetate zinc trisodium, pirbuterol acetate, revefenacin, ribavirin, salmeterol xinafoate, sevoflurane, terbutaline sulfate, tetrahydrocannabinol, cannabidiol, tiotropium bromide, tobramycin, trimcinolone acetonide, umeclidinium bromide, vilanterol trifenatate, xenon xe-133, zanamivir, epinephrine, sodium chloride, interferon beta, interferon beta 1-b, interferon beta gene delivery, interferon beta-1a, a BKB2R antagonist, a KLKB1 inhibitor, androgens, recombinant SERPING1, vitamin D, a HAS2 or HAS3 inhibitor, timbetasin, and combinations thereof.

In another aspect, a method is provided for regenerating or restoring respiratory tissue or respiratory function in a subject following an acute respiratory disorder in the subject, the method comprising administering, to lung tissue of the subject, a composition comprising amniotic fluid, an amnion tissue preparation, or a combination thereof. In some embodiments, the method can optionally include one or more of the following features. The administering can occur after acute treatment of a respiratory disorder. The acute treatment can comprise mechanical ventilation, ambulatory oxygen administration, or a combination thereof. The administering can occur after the subject has been discharged from hospital care, downgraded from intensive care, downgraded from acute care, downgraded from critical care, or removed from acute care treatment. The administering can occur more than 1 day, more than 2 days, more than 3 days, more than 1 week, more than 2 weeks, more than 3 weeks, more than 6 weeks, more than 8 weeks, more than 10 weeks, or more than 15 weeks after the subject has been discharged from hospital care, downgraded from intensive care, downgraded from acute care, downgraded from critical care, or removed from acute care treatment. The administering can include administering once daily, multiple times daily, every other day, weekly, or monthly for a period of from about 1 day to about 10 years following the acute treatment. The respiratory disorder can be selected from chronic obstructive pulmonary disease, asthma, acute asthma, chronic asthma, severe asthma, allergic asthma, bronchial asthma, intrinsic asthma, respiratory distress syndrome of the newborn, reversible respiratory disease, cystic fibrosis, bronchospasms, bronchitis, chronic bronchitis, bronchiectasis, alpha-1 antitrypsin emphysema, emphysema, associated cor pulmonale with pulmonary hypertension, right ventricular hypertrophy and right heart failure, pulmonary hypertension, interstitial lung disease, pulmonary fibrosis, pneumonia, interstitial pneumonia, a lung infection, idiopathic pulmonary fibrosis, cystic fibrosis, tuberculosis, severe acute respiratory syndrome, infection, pulmonary embolus, pulmonary arterial hypertension, pulmonary edema, pneumocystis pneumonia, SARS-CoV-2 infection, covid-19, coronavirus, acute respiratory distress syndrome, intensive care unit (ICU) syndrome, systemic inflammatory response syndrome (SIRS), sepsis, severe sepsis, septic shock, or multiple organ dysfunction syndrome (MODS), cystic fibrosis, sarcoidosis, and combinations thereof, and wherein the non-respiratory disorder is selected from an autoimmune disease, a spondyloarthropathy, an intestinal disease, diabetes, a skin disease, a non-respiratory infection, a pain disorder, intensive care unit (ICU) syndrome, systemic inflammatory response syndrome (SIRS), sepsis, severe sepsis, septic shock, or multiple organ dysfunction syndrome (MODS), cystic fibrosis, sarcoidosis, and combinations thereof. The composition can further comprise stem cells, a stem cell preparation, or combinations thereof. The composition can further comprise one or more active agents selected from acetyl cysteine, aclidinium bromide, albuterol, albuterol sulfate, amikacin sulfate, an amnion tissue preparation, arformoterol sulfate, atropine sulfate, aztreonam, beclomethasone dipropionate, bitolterol mesylate, budesonide, ciclesonide, cromolyn sodium, desflurane, dexamethasone sodium phosphate, dornase alfa, enflurane, epinephrine, ergotamine tartrate, flunisolide, fluticasone propionate, fomoterol fumarate, glycopyrrolate, halothane, indacaterol maleate, iloprost, insulin, ipratropium bromide, isoetharine hydrochloride, isoflurane, isoproterenol hydrochloride, levalbuterol hydrochloride, levodopa, loxapine, mannitol, metaproterenol sulfate, methacholine chloride, mometasone furoate, nedocromil sodium, nicotine, nitric oxide, olodaterol hydrochloride, pentamidine isethionate, pentetate calcium trisodium, pentetate zinc trisodium, pirbuterol acetate, revefenacin, ribavirin, salmeterol xinafoate, sevoflurane, terbutaline sulfate, tetrahydrocannabinol, cannabidiol, tiotropium bromide, tobramycin, trimcinolone acetonide, umeclidinium bromide, vilanterol trifenatate, xenon xe-133, zanamivir, epinephrine, sodium chloride, interferon beta, interferon beta 1-b, interferon beta gene delivery, interferon beta-1a, a BKB2R antagonist, a KLKB1 inhibitor, androgens, recombinant SERPING1, vitamin D, a HAS2 or HAS3 inhibitor, timbetasin, and combinations thereof.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the description below. Other features, objects, and advantages of the invention will be apparent from the description, and from the claims.

### DETAILED DESCRIPTION

Provided herein are inhalable compositions comprising amniotic fluid or an amnion tissue preparation. Also provided herein are various breathing systems and devices, including ventilators, nebulizers, inhalers, vaping devices, and e-cigarettes, comprising an inhalable composition comprising amniotic fluid or an amnion tissue preparation. Typically, a composition provided herein (e.g., a composition containing amniotic fluid or an amnion tissue preparation, or a combination thereof) is administered via inhalation, and the composition will have a formulation or particle size suitable for delivery to the respiratory tract of a subject (e.g., a mammal such as a human, dog, cat, horse, cow, pig, sheep, goat, or monkey). This document also provides methods for using compositions (e.g., inhalable formulations) and devices described herein for treating or preventing respiratory disorders, methods of providing long-term maintenance treatment following an acute treatment of a respiratory disorder (including but not limited to acute respiratory disorders), and methods of regenerating or restoring respiratory tissue (including but not limited to lung tissue) or respiratory function. In some embodiments, the respiratory disorders and conditions can include bronchospasms, COPD, chronic bronchitis, asthma, emphysema, pulmonary hypertension, interstitial lung disease, pulmonary fibrosis, pneumonia, interstitial pneumonia, lung infections, idiopathic pulmonary fibrosis, covid-19, acute respiratory distress syndrome, and infections such SARS-CoV-2, SARS-CoV, MERS, and Pertussis. Methods of treatment described herein can include treatment of subjects requiring mechanical breathing assistance (e.g. mechanical ventilation), spontaneously breathing subjects with artificial airways, or ambulatory subjects capable of independent, spontaneous breathing.

The compositions described herein can be inhalable compositions that comprise amniotic fluid, an amnion tissue preparation, or combinations thereof.

In some embodiments, the composition can include amniotic fluid (“AF”).

AF is the fluid contained within the amniotic membrane, which forms a sac around the embryo and later fetus. AF, which is produced together by the fetus and the placenta, contacts the fetus, including the lung tissue, during the gestational period.

The composition of AF is, in some cases, not entirely known. However, AF can contain suspended amniocytes, stem cells, monocytes, macrophages, and histiocytes, as well as non-cellular components such as small molecules (including, but not limited to electrolytes, glutamine, arginine, and hyaluronic acid), growth factors (including, but not limited to, growth factor alpha (“TGF-α”), epidermal growth factor (“EGF”), insulin-like growth factor I (“IGF-1”), hyaluronic acid-stimulating factor, macrophage colony-stimulating factor (“M-CSF”), and granulocyte colony-stimulating factor (“G-CSF”)), and hormones (including, but not limited to, erythropoietin). AF can also contain immunomodulators and antimicrobials, including a-defensins, lactoferrin, lysozyme, bactericidal/permeability-increasing proteins, calprotectin, secretory leukocyte protease inhibitor, psoriasin, a cathelizidin, and various polyamines with antimicrobial properties. AF can also contain additional compounds or components that can provide benefits to a subject. Without being bound by any particular theory, it is believed that delivery of AF to the lungs of a subject having a respiratory disorder, including delivery to the lung tissue of the subject, can provide multiple potential benefits. For example, in some cases, components such as glutamine can aid in localized nucleic acid synthesis in the subject, and can lead to tissue regeneration. In some cases, components such as arginine can aid in regenerative angiogenesis in the lung tissue. In some cases, components such as hyaluronic acid can mitigate or reduce scaring and fibrosis in the lungs, for example, by inhibiting collagen synthesis. In some cases, AF components such as growth factors can stimulate proliferation of stem cells and non-progenitor cell-types in the subject's cells and tissues, including the lungs. In some cases, AF components such as erythropoietin can promote proliferation of red blood cell progenitors and may stimulate growth of endothelial cells and tissue. In some cases, antimicrobial components of AF can aid in destroying, reducing, or inhibiting lung infections and microbial growth in the lungs. In some cases, immunomodulators contained in AF can stimulate, suppress, or otherwise modulate the subject's immune response, and in particular, the subject's immune response within the lungs and lung tissue.

In some embodiments, the AF can provide healing or regeneration of lung tissue. In some embodiments, the AF can provide modulation of mediation of immune responses within the lung or lung tissue, thus halting or preventing damage to lung tissue caused directly or indirectly by a subject's immune response. In some embodiments, deposition of AF in the lower respiratory tract can allow for deposition and absorption of beneficial components in AF. In some embodiments, at least a portion of the AF can be deposited along and absorbed along the bronchial tree as it travels toward the lower respiratory tract.

In some embodiments, the AF is human AF. However, in some embodiments, AF from other mammalian species may also be used. For example, AF can be used from species including, but not limited to horse, rabbit, lamb, cow, sheep, primates, and the like.

AF can be obtained by any method known in the art. For example, human AF can, in some embodiments, be obtained from humans who are undergoing amniocentesis, humans who are undergoing a Caesarean section delivery, humans undergoing vaginal delivery using a specially designed receptacle to collect the fluid after rupture of membranes, and the like. In some embodiments, AF can be collected under sterile conditions in the operating room during an elective Cesarean section delivery since this form of collection presents essentially no risk to the infant or the mother. Similar methods can be used to obtain AF from other species. In some embodiments, the AF is collected under sterile conditions. In some embodiments, the AF can be further processed to sterilize or otherwise alter the AF. For example, in some embodiments, the AF can be processed in a manner that destroys viable cells contained within the AF, producing AF that lacks viable cells. For example, AF can be obtained and then treated in a manner designed to lyse some or all of the cells within the AF. In some embodiments, unaltered AF may be used. In some embodiments, the AF can contain viable cells, non-viable cells, or a combination thereof. In some embodiments, the AF can be screened for disease agents and other contaminants after collection and before use in a subject as described herein. For example, the AF can be screened for disease agents such as HIV, HTLV, Hepatitis B and C, syphilis, and the like. and other contaminants after collection and before use in a subject as described herein. For example, the AF can be screened for disease agents such as HIV, HTLV, Hepatitis B and C, syphilis, and the like. In some embodiments, an amnion tissue preparation can be sterile de-cellularized human amniotic fluid, either in fluid form or solid form (e.g., lyophilized powder), alone or in combination with appropriate excipients. Some exemplary methods of preparing sterile de-cellularized amniotic fluid are described in detail in, e.g., U.S. application Ser. No. 15/053,497, incorporated herein in its entirety.

In some embodiments, the AF is free of amniotic membrane or amniotic membrane particulate matter. For example, in some embodiments, the AF has been clarified or otherwise processed after collection to remove, for example, cellular debris from the amniotic membrane, but that retains macromolecules typically present in AF (e.g. proteins, lipids, nucleic acids, sugars, and the like). Standard techniques for removing particulate matter from biological samples can be used to remove the amniotic membrane particulate matter, including, but not limited to centrifugation (e.g. at a speed in the range of from about 1000 rpm to about 5000 rpm).

In some embodiments, the AF can be treated, to, e.g., provide preservation or lengthen shelf life, and the like. For example, in some embodiments, the AF can be treated by sterilization (e.g. by gamma-irradiation), or can be cooled by refrigeration or freezing. In some embodiments, substances may be added to the AF to, for example, prevent the growth of microbes (e.g. antifungal, antibacterial or antiviral agents). In some embodiments, the AF can be lyophilized (i.e. freeze-dried), stored, and then reconstituted for use as necessary. Standard lyophilization techniques can be used. In some embodiments, lyophilized AF can be reconstituted with, for example, physiologically compatible saline solutions. In some embodiments, the lyophilized AF can be reconstituted with AF, in circumstances where, for example, concentrated AF is desired. In some embodiments, the AF may can be concentrated by removal of water by any standard technique. For example, in some embodiments, essentially all water may be removed (e.g. by lyophilzation). In some embodiments, the amount of water may simply be reduced (e.g. by vacuum filtration, etc.). In some embodiments, the AF is undiluted AF. In some embodiments, a diluted or concentrated form of AF can be used. For example, compositions can include a concentration of from about 10% to about 200% AF, from about 10% to about 95% AF, from about 10% to about 90% AF, from about 20% to about 80% AF, from about 30% to about 70% AF, from about 40% to about 60% AF, from about 100% to about 200% AF, from about 110% to about 200% AF, from about 120% to about 190% AF, from about 130% to about 180% AF, from about 140% to about 170% AF, about 50% AF, about 60% AF, about 70% AF, about 80% AF, about 90% AF, about 100% AF, about 110% AF, about 120% AF, about 130% AF, about 140% AF, about 150% AF, about 160% AF, about 170% AF, about 180% AF, about 190% AF, or about 200% AF in the composition. In the case of a liquid composition, the dilution may be made with any of several suitable diluents that are known to those of skill in the art, for example, physiologically compatible saline solution, balanced saline solution, sodium hyaluronate, methylcellulose, and the like.

In some embodiments, the composition can include an amnion tissue preparation. Placental tissue comprises two major membrane components, the amnion and the chorion. The amnion layer is interior to the chorion in relation to the amniotic sac that encloses a mammalian, e.g., human, embryo. An “amnion tissue preparation” as used herein refers to a preparation of amnion tissue or amnion material, e.g. from the amnion layer of the amniotic sac, a portion thereof, or any material including or derived from the amnion layer. For example a preparation of amnion tissue or amnion material can include amniotic membrane (AM), an amniotic membrane extract, an amniotic membrane jelly extract, an amniotic membrane stromal extract, and the like. In some embodiments, the amnion layer of the amniotic sac can be separated from the chorion to be used as an amnion tissue preparation. Alternatively, in some embodiments, placental tissue, which contains both the amnion layer and the chorion layer, can be used to produce and amnion tissue preparation.

Amnion tissue preparations can be in the form of a liquid, suspension, or dried form (e.g., ground or pulverized lyophilized powder), or other forms. In some embodiments, the amnion tissue preparation can be in the form of a liquid. In some embodiments, the amnion tissue preparation can be in the form of a dried powder. In some embodiments, the amnion tissue preparation can be a dried amnion tissue preparation. In some embodiments, the amnion tissue preparation can be a dried powder suspended or dissolved in a liquid.

The term “liquid amnion tissue preparation” as used herein refers to a preparation of amnion tissue or amnion material that has a water content of at least 8.1%. In some embodiments, the liquid amnion tissue preparation can have a water, or other liquid content, that is greater than about 8.5% (e.g., greater than about 9%, greater than about 10%, greater than about 15%, greater than about 20%, greater than about 30%, greater than about 40%, greater than about 50%, greater than about 60%, greater than about 70%, greater than about 80%, or greater than about 90%). In some embodiments, an amnion tissue preparation can be a liquid preparation (e.g., solution or suspension) that is prepared from a dried amnion tissue preparation.

The term “dried amnion tissue preparation” as used herein refers to a preparation of amnion tissue or amnion material that is dried to have a water content that is less than about 8 percent (e.g., less than about 7%, less than about 6%, less than about 5%, less than about 4%, less than about 3%, less than about 2%, or less than about 1%). In some embodiments, a dried amnion tissue preparation can have a water content that is between about 0.1% and about 8% (e.g., between about 0.5% and about 8%, between about 1% and about 8%, between about 0.1% and about 5%, between about 0.1% and about 4%, between about 0.1% and about 3%, between about 0.5% and about 5%, or between about 1% and about 4%).

A dried amnion tissue preparation can be stored in a smaller volume, and may not require the same low temperature storage requirements to keep the formulation from degrading over time. In some embodiments, an amnion tissue preparation can be dried using any appropriate technique such as micronization, vacuum drying, spray drying, freeze drying, or combinations thereof In some embodiments, an amnion tissue preparation or stem cell preparation can be dried as described elsewhere (e.g., U.S. Pat. No. 5,656,498). A dried amnion tissue preparation can have any appropriate particle size for delivery via inhalation. For example, in some embodiments, a dried amnion tissue preparation, for example, a dried amnion tissue preparation for dissolution and reconstitution, can have a particle size ranging from about 0.1 μm to about 25 μm (e.g., from about 0.5 μm to about 25 μm, from about 0.75 μm to about 25 μm, from about 1 μm to about 25 μm, from about 0.1 μm to about 15 μm, from about 0.1 μm to about 10 μm, from about 0.1 μm to about 7.5 μm, from about 0.1 μm to about 5 μm, from about 0.75 μm to about 7.5 μm, or from about 1 μm to about 5 μm). In some embodiments, a dried amnion tissue preparation, for example, a dried amnion tissue preparation for dissolution, suspension, or direct inhalation can have a particle size ranging from about 0.1 μm to about 5 μm (e.g., from about 0.2 μm to about 5 μm, from about 0.5 μm to about 5 μm, from about 1 μm to about 5 μm, from about 1.5 μm to about 5 μm, from about 2 μm to about 5 μm, from about 2.5 μm to about 5 μm, from about 2.75 μm to about 5 μm, from about 3 μm to about 5 μm, from about 3.25 μm to about 5 μm, from about 3.5 μm to about 5 μm, from about 3.75 μm to about 5 μm, from about 4 μm to about 5 μm, from about 4.25 μm to about 5 μm, from about 4.5 μm to about 5 μm, from about 4.75 μm to about 5 μm, from about 0.1 μm to about 4.5 μm, from about 0.2 μm to about 4.5 μm, from about 0.5 μm to about 4.5 μm, from about 1 μm to about 4.5 μm, from about 1.5 μm to about 4.5 μm, from about 2 μm to about 4.5 μm, from about 2.5 μm to about 4.5 μm, from about 2.75 μm to about 4.5 μm, from about 3 μm to about 4.5 μm, from about 3.25 μm to about 4.5 μm, from about 3.5 μm to about 4.5 μm, from about 3.75 μm to about 4.5 μm, from about 4 μm to about 4.5 μm, from about 4.25 μm to about 4.5 μm). In some embodiments, dried amnion tissue preparation can be stored and reconstituted prior to use, or used directly as a dried amnion tissue preparation (e.g., dry powder amnion tissue preparation). In some embodiments, the dried amnion tissue preparation be stored. The storage temperature can vary from less than about −196° C.-80° C., −50° C., or −20° C. to more than about 23° C. If desired, the powder can be characterized (weight, protein content, etc.) prior to storage.

In some embodiments, the final composition containing a dried amnion tissue preparation may not be a dried composition or a dry powder composition. For example, in some embodiments, the composition may include liquid or fluid components that render the final composition a liquid or fluid. For example, compositions used in metered dose inhalers may include one or more of propellants, surfactants, flavorants, and the like. In some embodiments, the compositions can include a dried amnion tissue preparation in combination with inactive ingredients that render the inhalable composition a liquid, fluid, cream, or semi-solid. In some embodiments, the dried amnion tissue preparation, e.g., dry powder amnion tissue preparation, can be reconstituted in a suitable solution or buffer prior to use. Exemplary solutions include but are not limited to PBS, DMEM, and BSS. In some embodiments, the pH of the solution can be adjusted as needed. The concentration of the amnion tissue preparation can be varied as needed, depending on the subject's respiratory disorder, medical condition, and the like. In some procedures a more concentrated preparation can be useful, whereas in other procedures, a solution with a low concentration of amnion tissue preparation can be useful. Additional compounds can be added to the reconstituted amnion tissue preparation. Exemplary compounds that can be added to the reconstituted composition include but are not limited to pH modifiers, buffers, collagen, HA, antibiotics, surfactants, stabilizers, proteins, and the like.

An amnion tissue preparation, e.g., a liquid amnion tissue preparation or a dried amnion tissue preparation, can contain viable cells, non-viable cells, or a combination thereof. For example, an amnion tissue preparation can be a preparation of amnion tissue or amnion material having viable cells. In some embodiments, an amnion tissue preparation can be a solution or suspension of amnion tissue or amnion material having viable cells. In some embodiments, amnion tissue or amnion material can be obtained and then treated in a manner designed to lyse some or all of the cells within the amnion tissue or amnion material. For example, in some embodiments, an amnion tissue preparation can be a preparation of amnion tissue or amnion material where all the cells were removed, killed, or lysed such that the amnion tissue preparation lacks viable cells. In some embodiments, a dried amnion tissue preparation can be a preparation of amnion tissue or amnion material that was exposed to one or more physical and/or chemical treatments that killed, fixed, or lysed the cells of the amnion tissue or amnion material such that the amnion tissue preparation lacks viable cells. For example, in some embodiments, temperature (e.g., rapid freezing or rapid freezing-thawing), force and pressure, and/or electrical disruption can be used to kill or lyse cells within amnion tissue or amnion material to produce an amnion tissue preparation that lacks viable cells.

In some embodiments, a dried amnion preparation can be prepared from human amnion tissue. In some embodiments, human amnion tissue can be harvested, processed to remove, kill, or lyse cells or to remove blood, and dried to form a dried amnion tissue preparation. In some embodiments, human amnion tissue can be processed to remove blood prior to forming a dried amnion tissue preparation. In some embodiments, human amnion tissue can be processed without removing cells or blood prior to forming a dried amnion tissue preparation.

An example of an amnion tissue preparation includes, without limitation, a dried human amnion tissue preparation that lacks viable cells, a dried human amnion tissue preparation that includes viable cells, a liquid human amnion tissue preparation that lacks viable cells, and a liquid human amnion tissue preparation that includes viable cells. In some embodiments, an amnion tissue preparation, such as a dried amnion tissue preparation, can be obtained from MiMedX® or a tissue bank (e.g., a human tissue bank).

In some embodiments, the compositions can comprise amniotic fluid, an amnion tissue preparation, or a combination thereof, in combination with a stem cell preparation.

Stem cell preparations can be in the form of a liquid, suspension, or dried form (e.g., ground or pulverized lyophilized powder), or other forms. In some embodiments, the stem cell preparation can be in the form of a liquid. In some embodiments, the stem cell preparation can be in the form of a dried powder. In some embodiments, the stem cell preparation can be a dried stem cell preparation. In some embodiments, the stem cell preparation can be a dried powder suspended or dissolved in a liquid.

The term “liquid stem cell preparation” as used herein refers to a preparation of stem cells or stem cell material that has a water content of at least 8.1%. In some embodiments, the liquid stem cell preparation can have a water, or other liquid content, that is greater than about 8.5% (e.g., greater than about 9%, greater than about 10%, greater than about 15%, greater than about 20%, greater than about 30%, greater than about 40%, greater than about 50%, greater than about 60%, greater than about 70%, greater than about 80%, or greater than about 90%). In some embodiments, a stem cell preparation can be a liquid preparation (e.g., solution or suspension) that is prepared from a dried stem cell preparation.

The term “dried stem cell preparation” as used herein refers to a preparation of stem cell or stem cell material that is dried to have a water content that is less than about 8 percent (e.g., less than about 7%, less than about 6%, less than about 5%, less than about 4%, less than about 3%, less than about 2%, or less than about 1%). In some embodiments, a dried stem cell preparation can have a water content that is between about 0.1% and about 8% (e.g., between about 0.5% and about 8%, between about 1% and about 8%, between about 0.1% and about 5%, between about 0.1% and about 4%, between about 0.1% and about 3%, between about 0.5% and about 5%, or between about 1% and about 4%).

A dried stem cell preparation can be stored in a smaller volume, and may not require the same low temperature storage requirements to keep the formulation from degrading over time. In some embodiments, a stem cell preparation can be dried using any appropriate technique such as micronization, vacuum drying, spray drying, freeze drying, or combinations thereof. In some embodiments, a stem cell preparation can be dried as described elsewhere (e.g., U.S. Pat. No. 5,656,498). A dried stem cell preparation can have any appropriate particle size for dissolution, suspension, or delivery via inhalation. For example, in some embodiments, a dried stem cell preparation, for example, a dried stem cell preparation for dissolution and reconstitution, can have a particle size ranging from about 0.1 μm to about 25 μm (e.g., from about 0.5 μm to about 25 μm, from about 0.75 μm to about 25 μm, from about 1 μm to about 25 μm, from about 0.1 μm to about 15 μm, from about 0.1 μm to about 10 μm, from about 0.1 μm to about 7.5 μm, from about 0.1 μm to about 5 μm, from about 0.75 μm to about 7.5 μm, or from about 1 μm to about 5 μm). In some embodiments, a dried stem cell preparation, for example, a dried stem cell preparation for dissolution, suspension, or direct inhalation can have a particle size ranging from about 0.1 μm to about 5 μm (e.g., from about 0.2 μm to about 5 μm, from about 0.5 μm to about 5 μm, from about 1 μm to about 5 μm, from about 1.5 μm to about 5 μm, from about 2 μm to about 5 μm, from about 2.5 μm to about 5 μm, from about 2.75 μm to about 5 μm, from about 3 μm to about 5 μm, from about 3.25 μm to about 5 μm, from about 3.5 μm to about 5 μm, from about 3.75 μm to about 5 μm, from about 4 μm to about 5 μm, from about 4.25 μm to about 5 μm, from about 4.5 μm to about 5 μm, from about 4.75 μm to about 5 μm, from about 0.1 μm to about 4.5 μm, from about 0.2 μm to about 4.5 μm, from about 0.5 μm to about 4.5 μm, from about 1 μm to about 4.5 μm, from about 1.5 μm to about 4.5 μm, from about 2 μm to about 4.5 μm, from about 2.5 μm to about 4.5 μm, from about 2.75 μm to about 4.5 μm, from about 3 μm to about 4.5 μm, from about 3.25 μm to about 4.5 μm, from about 3.5 μm to about 4.5 μm, from about 3.75 μm to about 4.5 μm, from about 4 μm to about 4.5 μm, from about 4.25 μm to about 4.5 μm). In some embodiments, dried stem cell preparation can be stored and reconstituted prior to use, or used directly as a dried stem cell preparation (e.g., dry powder stem cell preparation). In some embodiments, the dried stem cell preparation be stored. The storage temperature can vary from less than about −196° C.-80° C., −50° C., or −20° C. to more than about 23° C. If desired, the powder can be characterized (weight, protein content, etc.) prior to storage.

In some embodiments, the final composition containing a dried stem cell preparation may not be a dried composition or a dry powder composition. For example, in some embodiments, the composition may include liquid or fluid components that render the final composition a liquid or fluid. For example, compositions used in metered dose inhalers may include one or more of propellants, surfactants, flavorants, and the like. In some embodiments, the compositions can include a dried stem cell preparation in combination with inactive ingredients that render the inhalable composition a liquid, fluid, cream, or semi-solid. In some embodiments, the dried stem cell preparation, e.g., dry powder stem cell preparation, can be reconstituted in a suitable solution or buffer prior to use. Exemplary solutions include but are not limited to PBS, DMEM, and BSS. In some embodiments, the pH of the solution can be adjusted as needed. The concentration of the stem cell preparation can be varied as needed, depending on the subject's respiratory disorder, medical condition, and the like. In some procedures a more concentrated preparation can be useful, whereas in other procedures, a solution with a low concentration of stem cell preparation can be useful. Additional compounds can be added to the reconstituted stem cell preparation. Exemplary compounds that can be added to the reconstituted composition include but are not limited to pH modifiers, buffers, collagen, HA, antibiotics, surfactants, stabilizers, proteins, and the like, as well as those listed herein for inclusion in the product substrate.

A stem cell preparation, e.g., a liquid stem cell preparation or a dried stem cell preparation, can contain viable cells, non-viable cells, or a combination thereof. For example, a stem cell preparation can be a preparation of stem cells or stem cell material having viable cells. In some embodiments, a stem cell preparation can be a solution or suspension of stem cell or amnion material having viable cells. In some embodiments, stem cells or stem cell material can be obtained and then treated in a manner designed to lyse some or all of the cells within the stem cell preparation or stem cell material. For example, in some embodiments, a stem cell preparation can be a preparation of stem cells or stem cell material where all the cells were removed, killed, or lysed such that the stem cell preparation lacks viable cells. In some embodiments, a dried stem cell preparation can be a preparation of stem cells or stem cell material that was exposed to one or more physical and/or chemical treatments that killed, fixed, or lysed the cells of the stem cell material such that the stem cell preparation lacks viable cells. For example, in some embodiments, temperature (e.g., rapid freezing or rapid freezing-thawing), force and pressure, and/or electrical disruption can be used to kill or lyse cells within stem cell material to produce a stem cell preparation that lacks viable cells.

In some embodiments, a stem cell culture can be obtained and then treated in a manner designed to lyse all the stem cells. In these cases, the resulting material (e.g., cellular remnants from lysed stem cells) can be used directly as a liquid stem cell preparation that lacks viable cells, dried to form a dried stem cell preparation that lacks viable stem cells, or dried and then reconstituted to form a liquid stem cell preparation that lacks viable cells.

Examples of stem cell preparations include, without limitation, a lung stem cell preparation such as a lung epithelial progenitor cell preparation, a mesenchymal stem cell (MSC) preparation (e.g., a MSC preparation obtained from fat tissue or bone marrow), an umbilical cord blood stem cell preparation, an embryonic stem cell preparation, and a human induced pluripotent stem cell preparation.

In some embodiments, a stem cell preparation can be prepared from cultures of stem cells. In some embodiments, a stem cell preparation can be prepared by washing a culture of stem cells in saline (e.g., phosphate buffered saline) to remove culture medium, evaporating to remove wash medium, adding a solution (e.g., saline, water, or a water and sugar solution) to the resulting stem cell preparation, and, optionally, repeating the evaporation step. After the optional second evaporation step, the stem cell preparation can be formulated into a powder that can be used directly as a dried stem cell preparation or mixed with a liquid formulation to produce a liquid product substrate containing a stem cell preparation.

An example of a stem cell preparation includes, without limitation, a dried human stem cell preparation that lacks viable cells, a dried human stem cell preparation that includes viable cells, a liquid human stem cell preparation that lacks viable cells, and a liquid human stem cell preparation that includes viable cells. In some embodiments, a stem cell preparation, such as a dried stem cell preparation or a liquid stem cell preparation, can be obtained commercially from, e.g., Stemedica Cell Technologies, Inc.

In some embodiments, compositions comprising AF or an amnion tissue preparation as described herein can have a particle size suitable for delivery to the upper respiratory tract (URT). The URT includes the nose, sinuses, pharynx and larynx. For example, compositions comprising AF or an amnion tissue preparation as described herein having a particle size ranging from about 5 μm to about 25 μm, from about 5 μm to about 15 μm, or from about 5 μm to about 10 μm can be used to treat a disorder of the URT.

In some embodiments, compositions comprising AF or an amnion tissue preparation as described herein can have a particle size suitable for delivery to the lower respiratory tract (LRT), which includes the trachea, upper bronchi, and lungs, and be used to treat a lung disorder such as exercise-induced pulmonary hemorrhage. For example, compositions comprising AF or an amnion tissue preparation as described herein having a particle size ranging from about 0.1 μm to about 5 μm (e.g., from about 0.5 μm to about 5 μm, from about 0.75 μm to about 5 μm, from about 1 μm to about 5 μm, from about 0.1 μm to about 2 μm, from about 0.1 μm to about 1 μm, from about 0.1 μm to about 0.75 μm) can be used to treat a lung disorder including, but not limited to, pulmonary hemorrhage, acute respiratory distress syndrome, covid-19, interstitial pneumonia, and other disorders that can benefit from delivery of medication to the LRT.

In some embodiments, the compositions described herein can include a mixture of particles having a mixture of particle sizes suitable for delivery to both the URT and LRT.

In some embodiments, additional components can be added to the compositions described herein as desired. For example, antimicrobial agents such as antibiotics or anti-fungal agents may be added. Other substances can be added to the compositions to stabilize and/or preserve the compositions. For example, agents can be added such as those that promote healing (e.g. vitamins), improve delivery of the AF or amnion tissue preparation or combination thereof to the lungs or otherwise enhance the delivery of the AF or amnion tissue preparation or combination thereof or treatment of the subject (e.g. carriers, propellants, salts, preservatives, colorants, and the like). Such additions may be made, so long as the compounds do not cause irritation of the lung, and do not interfere with the desirable action of the AF or amnion tissue preparation. The compositions can be packaged and stored, for example, at room temperature, or for example, at 0° C. to 4° C., −10° C. to −20° C., or −80° C. prior to use.

In some embodiments, a composition comprising AF or an amnion tissue preparation as described herein can include one or more additional therapeutic agents. For example, compositions described herein can include, without limitation, one or more bronchodilators, one or more anti-inflammatory agents (e.g., non-steroidal anti-inflammatory drugs, dexamethasone or other type of glucocorticoid steroids), one or more growth factors (e.g., platelet derived growth factor PDGF, epithelial growth factor (EGF), fibroblast growth factor-2 (FGF2), or stem cell factor (SCF)), one or more lung surfactants (e.g., DPPC), and/or one or more antimicrobial agents (e.g., antibiotics such as kanamycin, neomycin, streptomycin, or gentamicin or an antifungal agent). In some embodiments, compositions described herein can include, as one or more additional therapeutic agents (in addition to the amniotic fluid, amnion tissue preparation, or combination thereof), one or more agents selected from acetyl cysteine, aclidinium bromide, albuterol, albuterol sulfate, amikacin sulfate, arformoterol sulfate, atropine sulfate, aztreonam, beclomethasone dipropionate, bitolterol mesylate, budesonide, ciclesonide, cromolyn sodium, desflurane, dexamethasone sodium phosphate, dornase alfa, enflurane, epinephrine, ergotamine tartrate, flunisolide, fluticasone propionate, fomoterol fumarate, glycopyrrolate, halothane, indacaterol maleate, iloprost, insulin, ipratropium bromide, isoetharine hydrochloride, isoflurane, isoproterenol hydrochloride, levalbuterol hydrochloride, levodopa, loxapine, mannitol, metaproterenol sulfate, methacholine chloride, mometasone furoate, nedocromil sodium, nicotine, nitric oxide, olodaterol hydrochloride, pentamidine isethionate, pentetate calcium trisodium, pentetate zinc trisodium, pirbuterol acetate, revefenacin, ribavirin, salmeterol xinafoate, sevoflurane, stem cells, a stem cell preparation, terbutaline sulfate, tetrahydrocannabinol, cannabidiol, tiotropium bromide, tobramycin, trimcinolone acetonide, umeclidinium bromide, vilanterol trifenatate, xenon xe-133, zanamivir, epinephrine, sodium chloride, and combinations thereof.

In some embodiments, the compositions described herein can include one or more pharmaceutically acceptable carriers or excipients. For example, carriers and excipients can include, without limitation, propellants, surfactants, solvents, preservatives, and flavorants. In some embodiments, the compositions can consist essentially of amniotic fluid, an amnion tissue preparation, or a combination thereof “Consisting essentially of” or “consists essentially of” can be used to indicate that only the specified active agents are present in the formulation and no other active pharmaceutical agents are present in the formulation, but inactive ingredients such as excipients can be present. For example, in some embodiments, a composition consisting essentially of amniotic fluid, an amnion tissue preparation, or combinations thereof, or a composition can include only the amniotic fluid, amnion tissue preparation, or combinations thereof as active agent, but can additionally include excipients or inactive ingredients described herein. In some embodiments, the compositions can consist of amniotic fluid, an amnion tissue preparation, or a combination thereof. In some embodiments, the compositions can consist essentially of amniotic fluid, an amnion tissue preparation, or combinations thereof, and stem cells, a stem cell preparation, or combinations thereof. “Consisting of” and “consist of” indicate that the amniotic fluid, amnion tissue preparation, or combination thereof is the sole ingredient, though water may be included in some embodiments for reconstitution or dilution.

In some embodiments, a product substrate liquid or solution can comprise one or more active agents and one or more pharmaceutically acceptable excipients, such as water, solvents, diluents, pH modifying agents, preservatives, antioxidants, suspending agents, wetting agents, viscosity modifiers, tonicity agents, stabilizing agents, and combinations thereof. Suitable pharmaceutically acceptable excipients are preferably selected from materials which are generally recognized as safe (GRAS) for use in respiratory administration.

In some embodiments, product substrate solutions can include pH adjusting agents or buffers to maintain a desired pH for the product substrate for storage, delivery into a subject, or a combination thereof. In some embodiments, the desired pH can be based on the conditions necessary to maximize stability of an active agent in the product substrate. In some embodiments, the product substrate or substrates can have a pH ranging from 3.5 to 10.0. In some embodiments, the product substrate or substrates can have a pH ranging from 5.5 to 8.5. In some embodiments, the product substrate or substrates can have a pH ranging from about pH 4.0 to about pH 8.5, from about pH 4.5 to about pH 7.5, from about pH 5.0 to about pH 6.5, from about pH 5.6 to about pH 6.3, from about pH 5.7 to about pH 6.2. In some embodiments, suitable pH values for the product substrate or substrates include about 4.0, about 4.5, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6. About 6.7, about 6.8, about 6.9, about 7.0, about 7.1, about 7.2, about 7.3, about 7.4, about 7.5, or about 8.5. In some embodiments, the pH of the product substrate or substrates is about 5.8. Suitable buffers include buffers generally recognized as safe (GRAS) for usage in lung tissue. Exemplary suitable buffers can include buffers selected from acetate, borate, carbonate, citrate, succinate, and phosphate buffers. In some embodiments, the buffer can be sodium citrate/citric acid. Alternatively, imidazole or histidine or another base or acid that maintains the pH in the range of about pH 3.0 to about 10.0 or about pH 4.5 to about pH 8.5 can be used.

In some embodiments, product substrate solutions can have a tonicity equivalent to a 0.5-7.0% solution of sodium chloride. In some embodiments, the product substrate solutions can be isotonic (e.g., have a tonicity equal to that of a 0.9% solution of sodium chloride). In some embodiments, product substrate solutions can include one or more tonicity agents to adjust the tonicity range of the substrate formulation. Exemplary suitable tonicity agents can include tonicity agents selected from glycerin, mannitol, sorbitol, sodium chloride, and other electrolytes.

In some embodiments, product substrate solutions can contain one or more preservatives, e.g., to prevent bacterial contamination. Non-limiting exemplary suitable preservatives include polyhexamethylenebiguanidine (PHMB), benzalkonium chloride (BAK), stabilized oxychloro complexes, phenylmercuric acetate, chlorobutanol, sorbic acid, chlorhexidine, benzyl alcohol, parabens, thimerosal, and the like, and combinations thereof.

In some embodiments, a product substrate solutions can contain one or more surfactants to facilitate inhalation or absorption of the products generated by the devices described herein. Non-limiting exemplary surfactants polyoxyethylene sorbitol esters such as polysorbate 80 (Tween 80) and polysorbate 20 (Tween 20); propylene-polyoxyethylene esters such as poloxamer 188, polyoxyethylene alcohols such as Brij35, mixtures of polysorbate surfactants with phospholipids such as phosphatidylcholine and derivatives (e.g., dipalmitoyl, dioleoyl, dimyristyl, or 1-palmitoyl), phospholipid glycerols such as dimyristol glycerol, lysophosphatidylcholine and derivatives thereof, lysolecithin, a mixture of polysorbate with cholesterol, a mixture of polysorbate surfactant with sorbitan surfactant (such as sorbitan monooleate, dioleate, trioleate, and the like), poloxamer surfactants, and combinations thereof. In some embodiments, the product substrate or product substrate solution can be free of surfactant.

In some embodiments, product substrate solutions can contain one or more pharmaceutically acceptable excipients or carriers such as dispersing agents, wetting agents, stabilizing agents, suspending agents, adjuvants, preservatives, flavorants, lipids, amino acids, surfactants, polymers, absorption enhancers, and the like, or combinations thereof. Exemplary excipients for use as stabilizing agents can include any sugar or sugar alcohol or any amino acid, such as, e.g., lactose, anhydrous lactose, mannitol, glucose, sucrose, trehalose, sorbitol, 1-O-alpha-D-glucopyranosyl-D-mannitol (e.g., Isomalt), xylitol, maltitol, lactitol, erythritol, arabitol, ribitol, fructose, mannose, galactose, raffinose, maltose, sorbose, cellobiose, inulin, sucrose, trehalose, raffinose, stachyose, sorbitol, dextrose, and combinations thereof. Additional optional excipients can include comprises one or more materials selected from an organic acid, organic base, polyol, peptide, protein, fat, fatty acid, amino acid (aspartic acid, glutamic acid, leucine, L-leucine, isoleucine, lysine, valine, methionine, phenylalanine, glycine, arginine, cysteine, alanine, serine, phenylalanine, lysine, N-acetyl-L-cysteine or a pharmaceutically acceptable salt, solvate, hydrate, or polymorph thereof), carbohydrate (e.g. mannitol, sorbitol, xylitol, mal ita, lactitol, erythritol, arabitol, ribitol, glucose, fructose, mannose, galactose, lactose, sucrose, raffinose, maltose, sorbose, cellobiose, trehalose, maltodextrins, dextrans, inulin, 1-O-alpha-D-glucopyranosyl-D-mannitol (Isomalt)), or their pharmaceutically acceptable solvate, hydrate or polymorph, phospholipid, triglyceride, detergent, polymer, sodium citrate, sodium ascorbate, lecithin, soya lecithin, dipalmitoylphosphatidyl diphospholidilipholina, ethanolamine, dipalmitoylphosphatidylinositol, phosphatidylcholines, phosphatidylethanolamine, phosphatidylglycerols, phosphatidylinositol, phosphatidylserine, sodium lauryl sulfate, magnesium lauryl sulfate; PEG 6000, PEG 3000 Tween 80, Poloxamer 188, leucine, L-leucine, isoleucine, lysine, valine, methionine, phenylalanine, glycine, arginine, aspartic acid, glutamic acid, cysteine, alanine, serine, or their pharmaceutically acceptable salt, solvate, hydrate or polymorph, and combinations thereof.

In some embodiments, the compositions described herein can be formulated into microparticles that contain solid lipid nanoparticles. In some embodiments, dry particles of AF or an amnion tissue preparation can be coated or encapsulated for delivery to an airway (e.g., a lung) via an aerosol based inhaler or a dry powder inhaler. Various respiratory devices can be used to deliver the compositions described herein to the lungs of a subject.

In some embodiments, a breathing system is provided herein, comprising an inhalable composition comprising amniotic fluid or an amnion tissue preparation. A breathing system is a medical system used to deliver inhalation assistance, and optionally, one or more inhalable medications. For example, a breathing system can deliver inhalation assistance by providing pressure assistance, oxygen delivery (including oxygen or a gas containing oxygen), carbon dioxide removal, or a combination thereof. In some embodiments, a breathing system can include one or more elements, such as one or more tubes, a source of gas flow, one or more valves (such as an adjustable pressure-limiting valve), a reservoir bag, a patient interface device (including but not limited to nasal prongs or a cannula, nasopharyngeal tubes or prongs, an endotracheal tube, a tracheostomy tube, a mask) and the like. In some embodiments the breathing system is an invasive system (e.g., using an endotracheal tube or a tracheostomy tube for assisted breathing), such as in patients that require full mechanical breathing assistance, or in patients under anesthesia. In some embodiments, the breathing system is a non-invasive system (e.g., systems using nasal prongs or masks). In some embodiments, the breathing system can include a breathing circuit. For example, in some embodiments, the breathing system can comprise a ventilator circuit.

In some embodiments, the breathing system comprises a breathing device (e.g., a pressure-assisted breathing device or other device for providing inhalation assistance). In some embodiments, the breathing system comprises a pressure-assisted breathing device. In some embodiments, the pressure-assisted breathing device can be selected from the group consisting of a mechanical ventilator, a continuous positive airway pressure system (“CPAP”), a bi-level positive airway pressure system (“BiPAP”), an automatic positive airway pressure system (“APAP”), and an adaptive servo ventilation system (“AVS”). A pressure-assisted breathing device is a device used for artificial ventilation that applies pressure to gases in or around a subject's airway to move, or assist in moving, gases into the lungs, out of the lungs, or a combination thereof. A pressure-assisted breathing device can increase or maintain lung volume, decrease the work of breathing for a subject, expand or prevent collapse of the subject's airways. Pressure, usually positive pressure, can be applied during or to assist in inhalation, or, in some embodiments, pressure can be applied during or to assist in exhalation, and combinations thereof.

In some embodiments, the breathing system can be an open system, a closed system, a semi-open system, or a semi-closed system. Open systems and semi-open systems use ambient air as the gas source and do not provide for rebreathing. An open system is unrestricted and provide no boundary between the subject's airway and the atmosphere. Semi-open systems utilize a restriction means, such as a reservoir or valve, that provides a partial boundary between the subject's airway and the atmosphere. Closed and semi-closed systems utilize a controlled gas source, such as oxygen or a non-ambient gas blend containing oxygen. A semi-closed system, such as a Mapelson A, B, C, D, E, or F system or a Humphrey ADE system, provides a full boundary between the subject's airway and the atmosphere for intake but allows venting of excess fresh gas into the atmosphere such that only partial rebreathing of exhaled air is possible. A closed system, such as a circle system, provides a fully closed boundary between the subject's airway and the atmosphere, preventing both air intake and venting and thus requiring complete rebreathing of exhaled gas.

In some embodiments, a breathing system is provided herein, comprising a pressure-assisted breathing device and an inhalable composition comprising amniotic fluid or an amnion tissue preparation. In some embodiments, the pressure-assisted breathing device can be a mechanical ventilator, such as a positive pressure ventilator, a negative pressure ventilator, or an intermittent abdominal pressure ventilator. Modes of mechanical ventilation can include volume modes (such as assist-control ventilation and synchronized intermittent-mandatory ventilation), pressure modes (such as pressure-controlled ventilation, pressure support ventilation, pressure controlled inverse ratio ventilation, and airway pressure release ventilation), dual modes (such as pressure regulated volume control), interactive modes (such as proportional assist ventilation and neurally adjusted ventilatory assist ventilation), inverse ratio ventilation, adaptive support ventilation, tube compensation, prone ventilation, high frequency oscillatory ventilation, high frequency percussive ventilation, positive end expiratory pressure, and combinations thereof. In some embodiments, a breathing system is provided herein, comprising a mechanical ventilator and an inhalable composition comprising amniotic fluid or an amnion tissue preparation. In some embodiments, the mechanical ventilator is an invasive mechanical ventilator. Examples of invasive mechanical ventilators include, but are not limited to, transport or mobile ventilators, intensive care ventilators, bubble ventilators, and neonatal ventilators. In some embodiments, a breathing system is provided herein, comprising a non-invasive mechanical ventilation device, and an inhalable composition comprising amniotic fluid or an amnion tissue preparation. Examples of non-invasive mechanical ventilation devices include, but are not limited to, a CPAP, a BiPAP, an APAP, or an ASV. In some embodiments, breathing system is provided herein, comprising a non-invasive mechanical ventilation device selected from a CPAP, a BiPAP, an APAP, or an ASV, and an inhalable composition comprising amniotic fluid or an amnion tissue preparation.

Breathing systems described herein can include accessory devices, such as gas heating devices, gas humidifying devices, pressure regulators, pressure monitors, alarm systems, microprocessors, valves such as one-way valves, reservoirs such as gas reservoirs, and devices and structures for the introduction of inhalable medications into the breathing system.

Devices and structures for introducing inhalable medications into the breathing system can include, without limitation, nebulizers, metered dose inhalers (such as pressurized metered dose inhalers and dry powder inhalers), tube joints, valves, spacers, and the like.

In some embodiments, a nebulizer is provided, comprising an inhalable composition comprising amniotic fluid or an amnion tissue preparation. In some embodiments, the nebulizer can be a nebulizer used for delivering an inhalable composition to an ambulatory subject that does not require mechanical breathing assistance. In some embodiments, the nebulizer can be a nebulizer used for delivering an inhalable composition to a subject that requires mechanical breathing assistance, including non-ambulatory subjects. For example, the nebulizer can, in some embodiments, be a nebulizer that can be operably connected to a breathing system that includes a pressure-assisted breathing device. In some embodiments, the nebulizer can be configured to directly interface with a subject, such as in an in-home setting in which a subject uses an interface such as a mask to deliver an inhalable composition such as those described herein to the subject without a pressure-assisted breathing device. In some embodiments, a breathing system can include a nebulizer comprising an inhalable composition comprising amniotic fluid, an amnion tissue preparation, or combinations thereof.

Nebulizers are drug delivery devices that are used for delivering compositions, typically in liquid form, to a subject's lungs in the form of a mist or aerosol that can be inhaled directly into the lungs. Exemplary nebulizers can include, but are not limited to, pneumatic nebulizers (e.g., jet nebulizers), mechanical nebulizers (e.g., soft mist inhalers), and electrical nebulizers (e.g., ultrasonic wave nebulizer, vibrating mesh nebulizer, and the like). Nebulizers described herein can include stationary or mobile nebulizers.

Additional substances can be included in the nebulizer compositions, such as excipients that improve the aerosolization of the compositions, flavorants, surfactants, and other excipients. Additional medicaments may also be included in the nebulizer formulations. For example, in some embodiments, a nebulizer can comprise a first medicament selected from AF, an amnion tissue preparation, or combinations thereof, and a second medicament. The second or additional medicament can be a drug, hormone, or other treatment. The amount of medicament(s) in the nebulizer can be determined by the required dose, which can be determined by, e.g., a physician. Suitable drugs for use as a second or additional medicaments include those for the treatment of respiratory disorders, e.g., bronchodilators, anti-inflammatories (e.g. corticosteroids), anti-allergics, anti-asthmatics, anti-histamines, and anti-cholinergic agents. Therapeutic proteins and peptides may also be employed for delivery by inhalation as second or additional medicaments. Exemplary drugs which may be employed for delivery by inhalation as second or additional medicaments include but are not limited to: albuterol, terbutaline, ipratropium, oxitropium, tiotropium, beclomethasone, flunisolide, budesonide, mometasone, ciclesonide, cromolyn sodium, nedocromil sodium, ketotifen, azelastine, ergotamine, cyclosporine, salmeterol, fluticasone, formoterol, procaterol, indacaterol, TA2005, omalizumab, zileuton, insulin, pentamidine, calcitonin, leuprolide, alpha-1-antitrypsin, interferons, triamcinolone, and pharmaceutically acceptable salts and esters thereof such as albuterol sulfate, formoterol fumarate, salmeterol xinafoate, beclomethasone dipropionate, triamcinolone acetonide, fluticasone propionate, tiotropium bromide, leuprolide acetate and mometasone furoate.

In some embodiments, a breathing system can include an inhaler comprising an inhalable composition comprising amniotic fluid or an amnion tissue preparation. In some embodiments, an inhaler is provided, comprising an inhalable composition comprising amniotic fluid or an amnion tissue preparation. In some embodiments, the inhaler can be an inhaler used for delivering an inhalable composition to an ambulatory subject that does not require mechanical breathing assistance. In some embodiments, the inhaler can be an inhaler used for delivering an inhalable composition to a subject that requires mechanical breathing assistance, including non-ambulatory subjects. For example, the inhaler can, in some embodiments, be an inhaler that can be operably connected to a breathing system that includes a pressure-assisted breathing device. In some embodiments, a breathing system can include an inhaler comprising an inhalable composition comprising amniotic fluid or an amnion tissue preparation.

Exemplary inhalers can include a unit dose inhaler or a multiple dose inhaler. The term “unit dose inhaler” refers to an inhaler that delivers a single dose of a composition or formulation, such as a dry powder formulation by inhalation to a subject user. Typically, a unit dose inhaler contains a single container that holds or contains an inhalable formulation. It will be appreciated that in some cases, multiple unit doses are required to provide a user with a specified dosage or treatment protocol. The term “multiple dose inhaler” refers to an inhaler having two or more containers, each container comprising a pre-metered dose of an inhalable composition, and the inhaler delivers a single dose of the inhalable composition by inhalation at any one time.

As used herein a “unit dose” refers to a pre-metered formulation (e.g., a dry powder formulation) for inhalation. In some embodiments, a unit dose can be a single container having multiple doses of formulation that can be delivered by inhalation as metered single amounts. A unit dose cartridge/container can contain a single dose. In some embodiments, it can include multiple individually accessible compartments, each containing a unit dose. Inhalers can include metered dose inhalers and pressurized metered dose inhalers. Metered dose inhalers typically produce an aerosol and comprise a medicament or a combination of medicaments and suitable liquefied propellant, such as a propellant selected from the group consisting of HFA 134a, HFA 227 and mixtures thereof.

In some embodiments described herein, an inhaler, such as a metered dose inhaler or dry powder inhaler can comprise AF, an amnion tissue preparation, or a combination thereof as a medicament. Aerosol formulations for use in metered dose inhalers may, as desired or needed, comprise other excipients, such as surfactant, a co-solvent (e.g. ethanol), CO2, or a particulate bulking agent. The medicament, e.g., AF, an amnion tissue preparation, or combinations thereof, may, in some embodiments, be provided in particulate form (for example, having a median size in the range of 1 to 10 microns) suspended in the liquefied propellant. Alternatively the medicament, e.g., AF, an amnion tissue preparation, or combinations thereof, may, in some embodiments, be in solution (e.g. dissolved) in the formulation. In some embodiments, a combination of two or more medicaments can be included, and all the medicaments may be suspended or in solution or alternatively one or more medicaments may be suspended, while one or more medicaments may be in solution.

In some embodiments, an additional medicament can be included in an inhaler, such as a metered dose inhaler or a dry powder inhaler, as described herein. For example, in some embodiments, an inhaler can comprise a first medicament selected from AF, an amnion tissue preparation, or combinations thereof, and a second medicament. The second or additional medicament can be a drug, vaccine, DNA fragment, hormone or other treatment. The amount of medicament(s) in the inhaler can be determined by the required dose per puff and available valve sizes, which are typically 25, 50 or 63 microliters, but can include 100 microliters where particularly large doses are required, and can be determine by, e.g., a physician. Suitable drugs for use as a second or additional medicaments include those for the treatment of respiratory disorders, e.g., bronchodilators, anti-inflammatories (e.g. corticosteroids), anti-allergics, anti-asthmatics, anti-histamines, and anti-cholinergic agents. Therapeutic proteins and peptides may also be employed for delivery by inhalation as second or additional medicaments. Exemplary drugs which may be employed for delivery by inhalation as second or additional medicaments include but are not limited to: albuterol, terbutaline, ipratropium, oxitropium, tiotropium, beclomethasone, flunisolide, budesonide, mometasone, ciclesonide, cromolyn sodium, nedocromil sodium, ketotifen, azelastine, ergotamine, cyclosporine, salmeterol, fluticasone, formoterol, procaterol, indacaterol, TA2005, omalizumab, zileuton, insulin, pentamidine, calcitonin, leuprolide, alpha-1-antitrypsin, interferons, triamcinolone, and pharmaceutically acceptable salts and esters thereof such as albuterol sulfate, formoterol fumarate, salmeterol xinafoate, beclomethasone dipropionate, triamcinolone acetonide, fluticasone propionate, tiotropium bromide, leuprolide acetate and mometasone furoate.

In another aspect, this document provides methods and materials for treating respiratory disorders and conditions. In some embodiments, the compositions, methods, breathing systems, and devices described herein can be used to treat a subject having one or more respiratory disorders, provide prophylaxis to a subject to prevent or reduce the severity of a developing respiratory disorder, provide maintenance treatment to a subject following an acute treatment of a respiratory disorder in the subject, or regenerate or restore respiratory tissue or respiratory function in a subject following an acute respiratory disorder. Exemplary respiratory disorders that can be treated, reduced, prevented, or repaired by administration of a composition that includes AF, an amnion tissue preparation, or a combination thereof (e.g., and inhalable composition) as described herein can include, without limitation, respiratory disorders or non-respiratory disorder.

In some embodiments of the methods described herein, the disorder is a respiratory disorder. In some embodiments, a respiratory disorder can include a disorder that manifests in both the respiratory system and other organ systems of body areas.

The respiratory disorder can include, without limitation, any obstructive lung disorders, and restrictive lung disorders. In some embodiments, the inhalable products or methods of treatment described herein can improve exercise endurance, increasing baseline blood oxygen saturation, reduce inflammation in the lungs of subjects with any obstructive lung disorders, and restrictive lung disorders, and the like. In some embodiments, the inhalable products or methods of treatment described herein can decrease subject dependency on use of other supplemental treatment such as bronchodilators, and/or oxygen therapy.

In some embodiments, the respiratory disorder can be selected from chronic obstructive pulmonary disease (COPD), asthma, acute asthma, chronic asthma, severe asthma, allergic asthma, bronchial asthma, intrinsic asthma (e.g., late asthma and airway hyper-responsiveness), respiratory distress syndrome of the newborn, reversible respiratory disease, cystic fibrosis, bronchospasms, bronchitis, chronic bronchitis, bronchiectasis, alpha-1 antitrypsin emphysema, emphysema, associated cor pulmonale (heart disease secondary to disease of the lungs and respiratory system) with pulmonary hypertension, right ventricular hypertrophy and right heart failure, pulmonary hypertension, interstitial lung disease, pulmonary fibrosis, pneumonia, interstitial pneumonia, lung infections, idiopathic pulmonary fibrosis, cystic fibrosis, tuberculosis, pneumonia, severe acute respiratory syndrome, infection, pulmonary embolus, tuberculosis, pulmonary arterial hypertension, pulmonary edema, pneumocystis pneumonia, covid-19, and acute respiratory distress syndrome. In some embodiments, the one or more active agents can be an active agent for treating or preventing lung injury related to systemic inflammatory response syndrome (SIRS), sepsis, severe sepsis, septic shock, and multiple organ dysfunction syndrome (MODS). In some embodiments, the one or more active agents can be an active agent for treating or preventing respiratory disorder such as a respiratory or respiratory-related infection. For example, in some embodiments, the one or more active agents can be an active agent for treating bacterial, fungal, or viral infections of the respiratory system. In some embodiments, the one or more active agents can be an active agent for treating infections such SARS-CoV-2, SARS-CoV, MERS, and Pertussis. In some embodiments, the one or more active agents can be an active agent for treating or preventing a lung injury, such as an acute inhalation injury, an injury from chemical irritants, asphyxiants, smoke, heat, riot control agents, chemical warfare agents, and similar exposures. In some embodiments, the one or more active agents can be a vaccine (e.g., a vaccine delivered through respiratory administration) for treating or preventing a respiratory disorder.

In some embodiments, the disorder is acute respiratory distress syndrome (ARDS). ARDS is a rapidly progressive disease occurring in critically ill patients and leads to complications such as leaking of fluid into the lungs. ARDS is sometimes initially diagnosed as pneumonia or pulmonary edema (fluid in the lungs from heart disease), and symptoms can include shortness of breath or severe shortness of breath, cough, fever, fast heart rates, rapid breathing, chest pain. In some embodiments, the methods described herein comprise treating, alleviating, or preventing one or more symptoms associated with acute respiratory distress syndrome.

In some embodiments, the disorder is pneumonia. Pneumonia is a common lung condition caused by bacterial, viral, or fungal infections, or by other inflammation of the lungs associated with, e.g., chemical exposure or the subject's immune response. Pneumonia symptoms can include cough (in some cases, coughing expels greenish or yellow mucus, or even bloody mucus), fever, shaking, chills, shortness of breath, and the like. In some embodiments, the methods described herein comprise treating, alleviating, or preventing one or more symptoms associated with pneumonia.

In some embodiments, the disorder is an idiopathic interstitial lung disease. Idiopathic interstitial lung disease can affect the lungs in at least three ways. First, lung tissue is damaged in a known or unknown manner. Second, the alveolar wall becomes inflamed. Third, scarring (or fibrosis) begins in the stroma (or tissue between alveoli), and the lungs harden. Examples of idiopathic interstitial lung disease include idiopathic pulmonary fibrosis (IPF). Idiopathic pulmonary fibrosis refers to a group of diseases characterized by deep lung tissue inflammation and eventual scarring resulting in shortness of breath. Scarring of the alveoli (alveolar sac) and its supporting structure (stroma) in IPF can result in a loss of functional alveolar units and ultimately reduces oxygen transport from the air to the blood. IPF is sometimes referred to as diffuse parenchymal lung disease, alveolitis, idiopathic fibrotic alveolitis (CFA), idiopathic pneumonia (IPP) and normal interstitial pneumonia (UIP). Subjects with IPF often exhibit symptoms such as dry cough, chest pain, or shortness of breath. In some embodiments, the inhalable products comprise prednisone, cytoxan, TNFα, combinations thereof, or other pulmonary agents. In some embodiments, the inhalable products and methods described herein comprise treating, alleviating, or preventing one or more symptoms associated with IPF (such as reducing or preventing pulmonary scarring).

In some embodiments, the disorder is a Chronic Obstructive Airway Disease (COAD). In COAD diseases, airflow obstruction can be chronic and persistent or incidental and recurrent. Airflow obstruction can be determined by forced expiratory spirometry, which records the volume of expiratory discharge during maximum expiration. In subjects whose airflow is not occluded, a complete forced expiration typically takes 3 to 4 seconds. In chronic obstructive airflow disorder patients with obstructed airflow, complete forced expiration typically takes up to 15 to 20 seconds and can be limited by hold-on time. A normal 1 second forced expiratory volume (FEV 1) is easily measured and accurately predicted based on age, gender and height. FEV 1 and the ratio of the forced vital capacity (FEV 1/FVC) is usually greater than 0.75. The recording of airflow versus volume during forced expiration and subsequent forced inspiration (flow-volume loop) is also useful mainly to distinguish upper airway stenosis from lower airway stenosis. Examples of chronic obstructive airway disease include asthma and chronic obstructive pulmonary disease (COPD).

In some embodiments, the disorder is asthma. Asthma generally includes disorders in which airway inflammation restricts airflow to and from the lungs. Asthma is also called bronchial asthma, exercise-induced asthma-bronchial and reactive airway disease (RAD). In some cases, asthma is associated with allergies or genetic background. Asthma is characterized by extensive short-term fluctuations in the diameter or inner diameter of the bronchial airways, including symptoms that result in changes in lung function. The resulting increased resistance to airflow results in symptoms in affected patients, including shortness of breath (dyspnea), chest tightness or “compression” and wheezing. Asthmatics are characterized according to NIH guidelines and expressed as mild intermittent, mild persistent, moderate persistent and severe persistent. Types of asthma can include asthma, acute asthma, chronic asthma, severe asthma, allergic asthma, bronchial asthma, intrinsic asthma (e.g., late asthma and airway hyper-responsiveness). In some embodiments, the inhalable products and methods described herein comprise treating, alleviating, or preventing one or more symptoms associated with asthma.

In some embodiments, the disorder is chronic obstructive pulmonary disease (COPD). COPD is typically characterized by inadequate airflow with varying degrees of alveolar enlargement and lung tissue destruction leading to irreversible airflow obstruction, and includes chronic bronchitis (hypersecretion with goblet cell submucosal hypertrophy), chronic obstructive bronchitis or emphysema (airway parenchyma destruction) or a combination of these conditions. In some embodiments, the inhalable products and methods described herein comprise treating, alleviating, or preventing one or more symptoms associated with COPD.

In some embodiments, the disorder is alpha-1 antitrypsin emphysema or emphysema. n some embodiments, the inhalable products and methods described herein comprise treating, alleviating, or preventing one or more symptoms associated with emphysema.

In some embodiments, the disorder is an acute inhalation injury. Inhaled substances can cause injury in the respiratory tract (e.g., in pulmonary epithelium). Chemical irritants, asphyxiants, toxic metals, products of fires and combustion, and many other substances can cause acute inhalation injury. Some cases of acute inhalation injury may involve more than one substance or mechanism. Some individuals are at increased risk of acute inhalation injury, including farmers working near silos, firefighters, coal miners, welders working with acetylene torches, military personnel, hockey rink workers, and chemical workers. Symptoms of acute inhalation injury can range from simple to severe. In some embodiments, the disclosed formulations are used for treating, alleviating, or preventing one or more symptoms associated with an acute inhalation injury.

In some embodiments, the disclosed formulations are used for treating, alleviating, or preventing one or more symptoms associated with an inhalation injury from a chemical irritant. Common exemplary chemical irritants include chlorine, hydrogen chloride, ammonia, hydrogen fluoride (HF) and hydrofluoric acid, sulphur dioxide (SO₂), nitrogen oxides, phosgene, hydrogen sulfide (H₂S).

In some embodiments, the disclosed formulations are used for treating, alleviating, or preventing one or more symptoms associated with an inhalation injury from an asphyxiant. Asphyxiants can include simple asphyxiants, which act by displacing oxygen from inspired air resulting in a reduced fraction of inspired oxygen and subsequent hypoxemia, and chemical asphyxiants (e.g., carbon monoxide and hydrogen cyanide), which act by interfering with oxygen delivery or utilization. Any gas in high concentration can act as an asphyxiant.

In some embodiments, the disclosed formulations are used for treating, alleviating, or preventing one or more symptoms associated with an inhalation injury from exposure to chemical warfare agents or riot control agents. Common exemplary warfare and riot control agents that can cause inhalation injury include Agent Orange, mustard gas, phosgene, chloropicrin, armamentarium, toxins derived from organophosphate pesticides, chloroacetophenone, orthochlorobenzamalonitrile, zinc chloride, and the like.

In some embodiments, the disclosed formulations are used for treating, alleviating, or preventing pulmonary edema.

In some embodiments, the disclosed formulations are used for treating, alleviating, or preventing one or more symptoms associated with an inhalation injury from toxic metals, such as cadmium and mercury.

In some embodiments, the disclosed formulations are used for treating, alleviating, or preventing one or more symptoms associated with burns or smoke inhalation, including exposure to heat, particulate matter, and toxic gases. In some embodiments, the disclosed formulations are used for treating, alleviating, or preventing one or more symptoms associated with smoke inhalation.

In some embodiments, the disclosed formulations are used for treating, alleviating, or preventing one or more symptoms associated with a blast injury.

In some embodiments, the disclosed formulations are used for treating, alleviating, or preventing one or more symptoms associated with an inhalation injury caused by complex exposure, such as exposure to more than one toxic compound.

In some embodiments, the disclosed formulations are used for treating, alleviating, or preventing one or more symptoms associated with an inhalation fever, such as metal fume fever, polymer fume fever, and organic dust toxic syndrome.

In some embodiments, the disclosed formulations are used for treating, alleviating, or preventing one or more symptoms associated with coalworker's pneumoconiosis.

In some embodiments, the disorder is a respiratory infection, such as a non-respiratory viral, bacterial, or fungal infection. In some embodiments, the disclosed formulations are used for treating, alleviating, or preventing one or more symptoms associated with a respiratory infection, such as SARS-CoV-2, SARS-CoV, MERS, tuberculosis, influenza, and Pertussis.

In some embodiments, the disorder is respiratory distress syndrome of the newborn, reversible respiratory disease, bronchospasms, bronchitis, bronchiolitis, chronic bronchitis, bronchiectasis, associated cor pulmonale (heart disease secondary to disease of the lungs and respiratory system) with pulmonary hypertension, right ventricular hypertrophy and right heart failure, pulmonary hypertension, interstitial lung disease, pulmonary fibrosis, pneumonia, interstitial pneumonia, lung infections, severe acute respiratory syndrome, pulmonary embolus, pulmonary arterial hypertension, pulmonary edema, pneumocystis pneumonia, and covid-19 (or SARS-CoV-2 infection) or combinations thereof.

In some embodiments of the methods described herein, the disorder is a disorder having both respiratory and non-respiratory symptoms.

In some embodiments, the disorder is intensive care unit (ICU) syndrome or ICU psychosis. ICU syndrome can occur subjects as psychotic episodes in intensive care units. In some cases, underlying causes include anxiety, sleep deprivation, sensory deprivation and overload, immobilization, unfamiliar environment, pain and the like. In some embodiments, the methods described herein comprise treating, alleviating, or preventing one or more symptoms associated with intensive care unit (ICU) syndrome.

In some embodiments, the disorder is systemic inflammatory response syndrome (SIRS), sepsis, severe sepsis, septic shock, or multiple organ dysfunction syndrome (MODS). These disorders are risk factors for the development of acute lung injury. In some embodiments, the methods described herein comprise treating, alleviating, or preventing one or more symptoms associated with systemic inflammatory response syndrome (SIRS), sepsis, severe sepsis, septic shock, or multiple organ dysfunction syndrome (MODS).

In some embodiments, the disorder is cystic fibrosis. Cystic Fibrosis (CF) is an inherited disease that causes thickened mucus to form in the lungs, pancreas and other organs. In some embodiments, the methods described herein comprise treating, alleviating, or preventing one or more symptoms associated with cystic fibrosis.

In some embodiments, the disorder is sarcoidosis. Sarcoidosis is a disease that causes overreaction of a subject's immune system. Sarcoidosis can lead to lung damage, skin rashes, and eye disease and can affect multiple organs of the body. In some embodiments, the methods described herein comprise treating, alleviating, or preventing one or more symptoms associated with sarcoidosis.

In some embodiments of the methods described herein, the disorder is a non-respiratory disorder. Active agents intended for systemic activity rather than pulmonary activity can be delivered by pulmonary administration, as the lung is capable of absorbing active agents for both local deposition and for systemic delivery. In some cases, systemic delivery via pulmonary administration can have advantages over other delivery routes because of faster absorption, lack of first-pass metabolism, and the like.

In some embodiments, the disorder is an autoimmune disease (e.g., rheumatoid arthritis, juvenile rheumatoid arthritis, and the like). In some embodiments, the disorder is a spondyloarthropathy (e.g., ankylosing spondylitis or psoriatic arthritis). In some embodiments, the disorder is an intestinal disease (e.g., Crohn's disease). In some embodiments, the disorder is diabetes (e.g., diabetes mellitus). In some embodiments, the disorder is a skin disease (e.g., psoriasis). In some embodiments, the disorder is a non-respiratory infection, such as a non-respiratory viral, bacterial, or fungal infection. In some embodiments, the one or more active agents can be an active agent for treating or preventing a non-respiratory disorder that is a pain disorder selected from neuropathic, nociceptive, acute, chronic and disease-specific pain (e.g., pain associated with osteoarthritis or fibromyalgia). In some embodiments, the methods described herein comprise treating, alleviating, or preventing one or more symptoms associated with an autoimmune disease (e.g., rheumatoid arthritis, juvenile rheumatoid arthritis, and the like), a spondyloarthropathy (e.g., ankylosing spondylitis or psoriatic arthritis), an intestinal disease (e.g., Crohn's disease), diabetes (e.g., diabetes mellitus), a skin disease (e.g., psoriasis), a non-respiratory infection (e.g., a non-respiratory viral, bacterial, or fungal infection), a pain disorder selected from neuropathic, nociceptive, acute, chronic and disease-specific pain (e.g., pain associated with osteoarthritis or fibromyalgia), and the like, and combinations thereof.

In some embodiments, the compositions comprising amniotic fluid, amnion tissue preparation, or combinations thereof can be delivered as inhalable products described herein to prevent a disease or disorder (e.g., a respiratory or non-respiratory disorder), such as part of inhalable products comprising a vaccine (e.g., a vaccine delivered through respiratory administration) for treating or preventing a non-respiratory disorder.

As described herein, disorders (e.g., respiratory disorders) can be treated by administering (e.g., via inhalation) an effective amount of a composition that includes AF, an amnion tissue preparation, or a combination thereof. Effective amounts of compositions described herein can be determined, e.g., by a physician, taking into account various factors such as overall health status, body weight, sex, diet, time and route of administration, other medications, and any other relevant clinical factors. As used herein, an “effective amount” or “therapeutically effective amount” of a composition is the amount that is sufficient to provide a beneficial effect to the subject to which the composition or preparations are delivered. The effective amount can be the amount effective to achieve an improved survival rate, a more rapid recovery, an improvement in the quality of life, or an improvement or elimination of one or more symptoms associated with a subject's respiratory disorder (e.g., covid-19).

In some embodiments, methods are provided herein for treating a subject or providing prophylaxis to a subject comprising mechanically ventilating the subject with a breathing system as described herein. In some embodiments, the methods can include delivering a composition described herein to the subject through the breathing system. For example, a method of treating a subject having a respiratory disorder or providing prophylaxis to a subject to prevent or reduce the severity of a developing respiratory disorder can include mechanically ventilating the subject with a breathing system, and delivering a composition to the subject through the breathing system, wherein the composition comprises amniotic fluid, an amnion tissue preparation, or a combination thereof. The breathing system can include a pressure-assisted breathing device, as described herein, such as a mechanical ventilator. In some embodiments, the pressure-assisted breathing device can be selected from the group consisting of an intensive care ventilator, a bubble ventilator, a continuous positive airway pressure system, a bi-level positive airway pressure system, an automatic positive airway pressure system, and an adaptive servo ventilation system. The breathing system can be configured to deliver the composition comprising amniotic fluid, an amnion tissue preparation, or a combination thereof to the subject. For example, in some embodiments, the breathing system can include a delivery device, such as a nebulizer, a metered dose inhaler, or a dry powder inhaler. The delivery device can be operably connected to the breathing system to deliver the composition to the lungs of the subject through the breathing system. For example, the delivery device can release an aerosol of the composition into the breathing system or aerosolize the composition in the breathing system, and the aerosol can be transported into the subject's lungs during ventilation (e.g., during inhalation). In some embodiments, the composition can be delivered to the subject as a liquid, a solution aerosol, a suspension aerosol, or a nebulized aerosol. The method can include actuating the delivery device to deliver one or more doses of the composition into the breathing system. Appropriate doses can be determined by, e.g., a physician, taking into account, the subject's condition, respiratory symptoms, age, weight, sex, height, BMI, oxygen saturation, or other circumstances.

The method can be useful for treatment or prophylaxis of any suitable respiratory disorder or condition (e.g., a respiratory disorder or a non-respiratory disorder). For example, respiratory disorders and conditions that can benefit from the methods described herein include, without limitation, bronchospasms, chronic obstructive airway disease, COPD, chronic bronchitis, asthma, emphysema, pulmonary hypertension, interstitial lung disease, pulmonary fibrosis, cystic fibrosis, pneumonia, interstitial pneumonia, lung infections (e.g., bacterial, viral, or fungal), idiopathic interstitial lung disease (e.g., idopathic pulmonary fibrosis), covid-19, acute respiratory distress syndrome, intensive care unit syndrome, acute inhalation injuries, and infections such SARS-CoV-2, SARS-CoV, MERS, and Pertussis. As another example, non-respiratory disorders and conditions that can benefit from the methods described herein include, without limitation, cardiovascular disease, an ocular disease, migraine, a pain-related disorder, an autoimmune disorder, alopecia, sexual dysfunction, skin treatment for psoriasis, and combinations thereof. Disorders having both respiratory and non-respiratory aspects and symptoms can also be treated using the methods described herein. For example, other disorders and conditions that can benefit from the methods described herein include, without limitation, cystic fibrosis, sarcoidosis, systemic inflammatory response syndrome (SIRS), sepsis, multiple organ dysfunction syndrome, and combinations thereof.

In some embodiments, methods are provided herein for treating a subject having a respiratory disorder, by administering, by way of ambulatory inhalation from a nebulizer, a composition comprising amniotic fluid, an amnion tissue preparation, or a combination thereof. In some embodiments, the composition can further comprise stem cells, a stem cell preparation, or combinations thereof. Ambulatory inhalation includes inhalation by a subject that can breathe without mechanical assistance. For example, a subject that can complete ambulatory inhalation can inhale and exhale without assistance. Such a subject could use the nebulizer at home, during a mobile situation, or at a clinic or hospital under (e.g., under physician supervision). In some embodiments, the nebulizer can be selected from a jet nebulizer, a soft mist nebulizer, an ultrasonic nebulizer, and a vibrating mesh nebulizer.

In some embodiments, methods are provided herein for treating a subject having a respiratory disorder by administering, to lung tissue of the subject, a composition comprising amniotic fluid, an amnion tissue preparation, or a combination thereof. In some embodiments, the composition can further comprise stem cells, a stem cell preparation, or combinations thereof. In some embodiments, the administering occurs through ambulatory inhalation of the composition by the subject from a delivery device. In some embodiments, the delivery device can be selected from a nebulizer, a metered dose inhaler, and a dry powder inhaler.

In some embodiments of the methods described herein, administration of the composition comprising AF, an amnion tissue preparation, and combinations thereof can occur after some initial treatment for a disorder (e.g., a respiratory disorder). For example, a subject may initially receive acute treatment for a disorder (such as a respiratory disorder or a non-respiratory disorder) in the form of, e.g. compositions described herein, or any other respiratory therapy or treatment (e.g., respiratory therapy or respiratory treatment). Then, some time after the initial treatment, such as, e.g., acute treatment, the subject can be administered one or more composition described herein. For example, after some period following acute treatment, a subject can receive a composition described herein as, e.g., a maintenance treatment, a regenerative treatment, a restorative treatment, or as prophylaxis for preventing relapse of a disorder or condition (e.g., a respiratory disorder). For example, in some embodiments, methods described herein can include providing maintenance treatment to a subject following an acute treatment of a respiratory disorder in the subject, by administering, to lung tissue of the subject, after completion of acute treatment of the subject's respiratory disorder, a composition comprising amniotic fluid, an amnion tissue preparation, or a combination thereof. As another example, in some embodiments, methods described herein can include regenerating or restoring respiratory tissue or respiratory function in a subject following an acute respiratory disorder in the subject by administering, to lung tissue of the subject, a composition comprising amniotic fluid, an amnion tissue preparation, or a combination thereof. In some embodiments, the composition can further comprise stem cells, a stem cell preparation, or combinations thereof. In some embodiments, acute treatment can include mechanical ventilation, oxygen administration, ambulatory oxygen administration, or a combination thereof. In some embodiments, administration of the compositions described herein after initial treatment can occur after the subject has been discharged from hospital care, downgraded from intensive care, downgraded from acute care, downgraded from critical care, or removed from acute care treatment. In some embodiments, administration of the compositions described herein after initial treatment can occur more than 1 day, more than 2 days, more than 3 days, more than 1 week, more than 2 weeks, more than 3 weeks, more than 6 weeks, more than 8 weeks, more than 10 weeks, or more than 15 weeks after the subject has been discharged from hospital care, downgraded from intensive care, downgraded from acute care, downgraded from critical care, or removed from acute care treatment. In some embodiments, administration of the compositions described herein after initial treatment can include administering once daily, multiple times daily, every other day, weekly, or monthly for a period of from about 1 day to about 10 years following the acute treatment.

In some embodiments, such as where a subject is provided with a maintenance treatment or prophylaxis against relapse of a respiratory disorder, the respiratory disorder can be selected from acute or chronic respiratory disorders, infections, and associated symptoms. Non-limiting examples of acute respiratory disorders include acute asthma, acute upper respiratory diseases (e.g., common cold or human coronavirus infections, common upper respiratory tract infections, influenza, diptheria, croup, allergic rhinitis, acute sinusitis, acute tonsilitis, acute pneumonia, pleural effusion, collapsed lung, acute bronchitis, bronciolitis, acute respiratory distress syndrome (ARDS), sudden acute respiratory syndrome (SARS, including SARS-CoV-2 or covid-19), pulmonary embolism, Middle East respiratory syndrome (MERS), pulmonary hypertension, acute pulmonary edema, respiratory depression (resulting from, e.g., opioid narcotics, barbituates, sedatives, alcohol, tumor, metabolic disorder, neuromuscular disease, airway obstruction, and the like), respiratory syncytial virus (RSV) infection, mucociliary dysfunction (e.g., resulting from acute infections including, but not limited to pertussis), cough, tuberculosis, acute interstitial lung disease, pulmonary hyperplasia, pulmonary interstitial emphysema, infant respiratory distress syndrome or surfactant deficiency disorder, and the like. Non-limiting examples of chronic respiratory disorders include chronic sinusitis, chronic epiglottitis, chronic pharyngitis, chronic stridor, chronic tonsillitis, chronic obstructive pulmonary disease (COPD) (including, but not limited to, chronic bronchitis, emphysema, bronchiolitis, non-reversible asthma, certain types of bronchiectasis), mucociliary dysfunction (e.g., resulting from chronic conditions including, but not limited to, cystic fibrosis (CF) and primary ciliary dyskinesia (PCD), chronic tuberculosis, cystic fibrosis, chronic pulmonary edema, neuromuscular disorders (including, but not limited to, myasthenia gravis, amyotrophic lateral sclerosis, and the like), primary ciliary dyskinesia, pulmonary MAC infection or MAC lung disease, interstitial lung disease (such as, but not limited to, idiopathic pulmonary fibrosis, nonspecific interstitial pneumonitis, and the like), chronic asthma, chronic symptoms or long-term periods of recovery from acute respiratory distress syndrome, Pseudomonas aeruginosa infection, respiratory cancers or tumors (including, but not limited to, primary carcinomas of the lung, small cell lung cancer, non-small cell lung cancer, (e.g., adenocarcinoma of the lung, squamous cell carcinoma of the lung, large cell lung carcinoma), carcinoid, Kaposi's sarcoma, melanoma, lymphoma, head and neck cancer, pleural mesothelioma, lung metastases of cancers (such as, but not limited to, breast cancer, liver cancer, colon cancer, prostate cancer, germ cell cancer, and renal cell carcinoma metastases), benign tumors (e.g., pulmonary hamartoma, pulmonary sequestration, and congenital cystic adenomatoid malformation (CCAM)), autoimmune disorders (such as, but not limited to, granulomatosis with polyangiitis, Goodpasture's syndrome, and the like), bronchopulmonary dysplaysia, and the like. In some embodiments, such as where the subject is provided with a method of regenerating or restoring respiratory tissue or respiratory function in a subject following an acute respiratory disorder in the subject, the respiratory disorder can be selected from ARDS, MERS, SARS, SARS-CoV-2 or covid-19, pneumonia, influenza, RSV infection, an inhalation injury, and the like.

In some embodiments, methods described herein can include delivering, to the subject, a composition comprising AF, an amnion tissue preparation, or a combination thereof in a form having a particle size range of from about 0.1 μm to about 5 μm (e.g., from about 0.2 μm to about 5 μm, from about 0.5 μm to about 5 μm, from about 1 μm to about 5 μm, from about 1.5 μm to about 5 μm, from about 2 μm to about 5 μm, from about 2.5 μm to about 5 μm, from about 2.75 μm to about 5 μm, from about 3 μm to about 5 μm, from about 3.25 μm to about 5 μm, from about 3.5 μm to about 5 μm, from about 3.75 μm to about 5 μm, from about 4 μm to about 5 μm, from about 4.25 μm to about 5 μm, from about 4.5 μm to about 5 μm, from about 4.75 μm to about 5 μm, from about 0.1 μm to about 4.5 μm, from about 0.2 μm to about 4.5 μm, from about 0.5 μm to about 4.5 μm, from about 1 μm to about 4.5 μm, from about 1.5 μm to about 4.5 μm, from about 2 μm to about 4.5 μm, from about 2.5 μm to about 4.5 μm, from about 2.75 μm to about 4.5 μm, from about 3 μm to about 4.5 μm, from about 3.25 μm to about 4.5 μm, from about 3.5 μm to about 4.5 μm, from about 3.75 μm to about 4.5 μm, from about 4 μm to about 4.5 μm, from about 4.25 μm to about 4.5 μm). For example, in some embodiments, the compositions can be delivered to the subject in an aerosol form, having a particle size of from about 3.5 μm to about 5 μm (e.g., from about 3.5 μm to about 5 μm, from about 3.75 μm to about 5 μm, from about 4 μm to about 5 μm, from about 4.25 μm to about 5 μm, from about 4.5 μm to about 5 μm, from about 4.75 μm to about 5 μm, from about 0.1 μm to about 4.5 μm, from about 0.2 μm to about 4.5 μm, from about 0.5 μm to about 4.5 μm, from about 1 μm to about 4.5 μm, from about 1.5 μm to about 4.5 μm, from about 2 μm to about 4.5 μm, from about 2.5 μm to about 4.5 μm, from about 2.75 μm to about 4.5 μm, from about 3 μm to about 4.5 μm, from about 3.25 μm to about 4.5 μm, from about 3.5 μm to about 4.5 μm, from about 3.75 μm to about 4.5 μm, from about 4 μm to about 4.5 μm, from about 4.25 μm to about 4.5 μm). In some embodiments, the compositions can be delivered to the subject in a non-aerosol form, such as a vapor form.

In some embodiments, methods described herein can include delivering, to the subject, a composition comprising AF made with from about 0.01 mg to about 1000 g (e.g., from about 0.01 mg to about 10 g, from about 0.1 mg to about 10 g, from about 1 mg to about 10 g, from about 10 mg to about 10 g, from about 100 mg to about 10 g, from about 1 g to about 100 g, from about 0.01 mg to about 5 g, from about 0.01 mg to about 1 g, from about 0.01 mg to about 100 mg, from about 10 mg to about 5 g, from about 100 mg to about 1 g, from about 10 g to about 100 g, from about 100 g to about 1000 g, or from about 1 g to about 5 g) of AF per kg body weight of the subject being treated. In some embodiments, the methods include delivering, to the subject, a composition comprising an amnion tissue preparation made with from about 0.01 mg to about 10 g (e.g., from about 0.01 mg to about 10 g, from about 0.1 mg to about 10 g, from about 1 mg to about 10 g, from about 10 mg to about 10 g, from about 100 mg to about 10 g, from about 1 g to about 10 g, from about 0.01 mg to about 5 g, from about 0.01 mg to about 1 g, from about 0.01 mg to about 100 mg, from about 10 mg to about 5 g, from about 100 mg to about 1 g, or from about 1 g to about 5 g) of amnion tissue per kg body weight of the subject being treated.

In some embodiments, the compositions containing AF, an amnion tissue preparation, or a combination thereof described herein can be delivered to the subject by inhalation only once. In some embodiments, multiple (e.g., two, three, four, five, six, seven, eight, nine, 10, 11, 12, 13, 14, 15, or 20 or more) deliveries of the compositions can be made by inhalation. For example, multiple deliveries of a composition containing AF, an amnion tissue preparation, or a combination thereof described herein can be made over the course of several (e.g., two, three, four, five, six, seven, eight, nine, 10, 14, 21, 28, or 31 or more) consecutive days (e.g., one delivery each day for seven days or one delivery every other day for seven days). In some embodiments, a composition containing AF, an amnion tissue preparation, or a combination thereof described herein can be delivered from about two to four times a day to about once per month. In some cases, a composition containing AF, an amnion tissue preparation, or a combination thereof described herein can be delivered to a subject for several months (e.g., one delivery per month for six months, one delivery per week for two months, from about one to about three deliveries per day for about four months, etc.),In some embodiments, deliveries of the compositions containing AF, an amnion tissue preparation, or a combination thereof described herein can be made as part of acute therapy, prophylactic therapy, maintenance therapy, therapeutic repair therapy, or regenerative therapy, depending upon the subject's condition, respiratory disorder, desired therapeutic goals, or temporal location in the progression of a respiratory disorder or condition.

In some embodiments, methods described herein can include identifying the subject as having or at risk of developing the respiratory disorder. In some embodiments, methods described herein can include identifying the respiratory disorder or one or more symptoms of the respiratory disorder. Identification of the subject, disorder, or symptoms can be conducted by any suitable manner, such as diagnostics, genetic analysis, lifestyle analysis, analysis of environmental conditions, and the like.

A composition containing AF, an amnion tissue preparation, or a combination thereof described herein can be delivered to a subject at various time points after diagnosis with a respiratory disorder (e.g., a lung infection), at various time points after indication that a subject is at risk of developing a respiratory disorder (e.g., after a subject has shown signs of early development of a respiratory disorder, or after a subject has been identified as having been exposed to an infectious disease that can cause a respiratory disorder, such as after exposure to SARS-CoV-2), at various time points after a subject has received treatment for an acute respiratory disorder (e.g., acute respiratory distress syndrome), or at various time points after a subject has exhibited some improvement (e.g., reduced symptoms, increased oxygen saturation, etc.) following treatment for a respiratory disorder. For example, a composition containing AF, an amnion tissue preparation, or a combination thereof described herein can be delivered immediately following diagnosis of a respiratory disorder (e.g., COPD), or immediately in cases of acute respiratory distress. In some cases, a composition containing AF, an amnion tissue preparation, or a combination thereof described herein can be delivered to a subject less than 10 (e.g., 9, 8, 7, 6, 5, 4, 3, 2, or 1) days after diagnosis with a respiratory disorder, after indication that a subject is at risk of developing a respiratory disorder, after a subject has received other treatment for an acute respiratory disorder, or after a subject has exhibited some improvement following treatment for a respiratory disorder.

The subject can be any mammal, e.g., a human (e.g., a human patient) or a non-human primate (e.g., chimpanzee, baboon, or monkey), a mouse, a rat, a rabbit, a guinea pig, a gerbil, a hamster, a horse, a type of livestock (e.g., cow, pig, sheep, or goat), a pangolin, a bat, a dog, or a cat. The subject can be any sex or age, including, e.g., neonatal, pediatric, young adult, adult, or geriatric. The subject can be a healthy subject. For example, in some embodiments, the methods, devices, and compositions described herein can be used to provide prophylactic treatment to a subject to prevent the development of a respiratory disorder, minimize the risk of developing a respiratory disorder, minimize the severity of a respiratory disorder that may develop in the future, improve lung capacity, or increase resistance to infection. In some embodiments, the subject can have an acute condition requiring acute treatment. For example, in some embodiments, the subject can be a subject having an acute infection affecting the respiratory tract, the subject can have a recently-diagnosed respiratory disorder, or the subject can be a subject receiving mechanical ventilation assistance or other life-supportive assistance for a condition or disorder that is a respiratory condition or disorder, a non-respiratory condition or disorder, or as part of a scheduled surgical or other procedure. In some embodiments, the methods, devices, and composition described herein can be used to prevent, minimize, reduce, or otherwise alleviate adverse respiratory effects of mechanical ventilation or life-supportive assistance. In some embodiments, the methods, devices, and composition described herein can be used in combination with mechanical ventilation or life-supportive assistance, to treat, reduce the severity of, or reduce one or more symptoms of a respiratory disorder. For example, in some embodiments, the methods, devices, and composition described herein can be used in conjunction with mechanical ventilation or life-supportive assistance to treat acute respiratory distress syndrome, an acute lung infection, or severe acute respiratory syndrome.

A composition described herein can be administered to a subject as a combination therapy with another treatment used to treat or prevent a respiratory disorder. For example, the combination therapy can include administering to the subject (e.g., a human patient) one or more additional agents that provide a therapeutic benefit to the subject who has, or is at risk of developing a respiratory disorder. In some cases, the composition and the one or more additional agents can be administered at the same time. In some cases, the composition can be administered first, and the one or more additional agents administered second, or vice versa (e.g., in a breathing circuit, in a nebulizer, with or without mechanical ventilation, etc.).

The efficacy of a given treatment in treating a particular respiratory disorder can be defined as an improvement of one or more symptoms of the respiratory disorder by at least 5% (e.g., at least 10%, at least 15%, at least 20%, at least 25%, at least 30%, at least 40%, at least 50%, at least 55%, at least 60%, at least 65% or more). In some cases, efficacy of a treatment with a composition containing a dried amnion tissue preparation and/or a dried stem cell preparation can be determined from the stabilization of one or more symptoms associated with the lung disorder (i.e., the treatments curtail the worsening of one or more symptoms of the lung disorder).

In some cases, the methods described herein can include monitoring the respiratory disorder in the subject to, for example, determine if the disorder is improving with treatment. Any appropriate method can be used to monitor a respiratory disorder. For example, for some subjects, lung function (e.g., using a spirometer or arterial blood gas test) can be monitored. For subjects diagnosed with exercise-induced pulmonary hemorrhage, clinical techniques designed to detect the presence of blood in lung airways can be used.

In some embodiments, a composition (e.g., an inhalable formulation) that includes AF or an amnion tissue preparation can be administered to humans who smoke tobacco products (e.g., cigarettes, cigars, or pipes) or to humans with a history of smoking tobacco products (e.g., cigarettes, cigars, or pipes) to reduce the severity of symptoms or respiratory disorders (e.g., lung symptoms) related to smoking or to reduce the development of symptoms or respiratory disorders(e.g., lung symptoms) related to smoking. For example, a human who smokes cigarettes can be administered a composition (e.g., an inhalable formulation) that includes AF or an amnion tissue preparation to reduce the severity of a chronic smoker's cough, a gravelly voice, and/or shortness of breath.

In some embodiments, compositions described herein can be used in an electronic vaping device or an electronic cigarette. In some embodiments, the compositions described herein can be used as a pre-vapor formulation in an electronic vaping device. In some embodiments, and electronic vaping device or electronic cigarette can be rechargeable, refillable, or disposable or single use.

In some embodiments, methods are provided herein for treating or preventing a respiratory condition comprising administering one of more compositions described herein to a subject via an electronic vaping device or electronic cigarette device. In some embodiments, the compositions described herein can be used in combination with, or before or after, another electronic vaping formulation such as another pre-vapor formulation. In some embodiments, methods are provided herein for treating or preventing a respiratory disorder caused by, induced by, or associated with electronic vaping or electronic cigarette use, comprising administering one of more compositions described herein to a subject via an electronic vaping device or electronic cigarette device. In some embodiments, the composition can include nicotine, one or more acids, one or more flavorants, or combinations thereof.

In some embodiments, the compositions and methods described herein can be used on mammalian subjects, and compositions described herein can be administered to mammalian subjects, including but not limited to humans, primates, canines, felines, bovines, equines, swine, rodents, and the like.

In some embodiments, a method of treating a mammal having exercise-induced pulmonary hemorrhage is provided. Exercise-induced pulmonary hemorrhage is a medical condition that refers to the presence of blood in lung airways in association with exercise. In some cases, between about 40 to 70 percent of horses may experience blood in the trachea following a horse race. Exercise-induced pulmonary hemorrhage (EIPH) is seen in most racehorses and in many other horses used in equine sports (e.g., polo, barrel racing, 3-day events) that require strenuous exercise for short periods of time, and between about 40 to 75 percent of horses may experience blood in the trachea or tracheobronchial tree (typically identified by endoscopic examination) following a horse race. Epistaxis is seen in a small proportion (˜5%) of horses with EIPH. In some cases, hemorrhage can be detected (e.g., by cytologic examination of bronchioalveolar lavage) in more than 90% of racehorses. EIPH has also been reported in human athletes and other mammals, such as racing camels and racing dogs, such as greyhounds. In some embodiments, the compositions and methods described herein can be used for, and compositions described herein can be administered for treating, alleviating, or preventing one or more symptoms associated with exercise induced pulmonary hemorrhage (EIPH) in mammals. In some embodiments, the mammal can be a human, a camel, a dog, or a horse. In some embodiments, the mammal can be a racing horse. In some embodiments, the compositions and methods described herein can be used for, and compositions described herein can be administered for treating, alleviating, or preventing one or more symptoms associated with epistaxis. In some embodiments, the method comprises, or consists essentially of, administering, to the mammal via inhalation, compositions described herein, in some embodiments, administered by the devices described herein. In some embodiments, the compositions can comprise AF, an amnion tissue preparation, or combinations thereof. In some embodiments, the compositions can further comprise stem cells, a stem cell preparation, or combinations thereof. In some embodiments, the compositions can consist essentially of AF, an amnion tissue preparation, or combinations thereof. In some embodiments, the compositions can comprise AF, an amnion tissue preparation, or combinations thereof, in combination with one or more other active agents (e.g., stem cells, a stem cell preparation, a bronchodilator, etc.).

In some embodiments, methods of managing or treating a pulmonary disease in equines, e.g., horses, comprising administering, using the compositions and methods described herein are provided. In some embodiments, a method of managing or treating a pulmonary disease in equines, e.g., horses, is provided, comprising administering, to an equine, compositions described herein by the devices described herein for treating, alleviating, or preventing one or more symptoms associated with a pulmonary disease in the equine. In some embodiments, the horses are racehorses. Airway diseases in horses typically present with symptoms such as coughing, nasal discharge, increased respiratory effort and poor performance or exercise intolerance. Fever, depression, decreased appetite, and weight loss can also be observed in horses with infectious airway diseases. In some embodiments, the equine pulmonary disease is selected from inflammatory airway disease or reactive airway disease (heaves). In some embodiments, the pulmonary disease is recurrent airway obstruction (RAO), e.g., previously known as chronic obstructive pulmonary disease (COPD). In some embodiments, the pulmonary disease is selected from viral respiratory infections such as equine herpesvirus infection, equine influenza, equine viral arteritis, and Hendra virus infection; secondary bacterial respiratory infections such as those caused by Streptococcus equi zooepidemicus, Actinobacillus equuli, Bordetella bronchiseptica, Escherichia coli, Pasteurella spp, Pseudomonas aeruginosa, or S equi equi, and resultant in mucosal bacterial infections (e.g., rhinitis and tracheitis) or resultant invasive disease (e.g., pneumonia and pleuropneumonia). In some embodiments, the formulations disclosed are suitable for treating, alleviating, or preventing one or more symptoms associated with rhinitis, tracheitis, pneumonia, or pleuropneumonia.

In some embodiments, methods of managing or treating a pulmonary disease in dogs and cats are provided. In some embodiments, the compositions and methods described herein can be used for, and compositions described herein can be administered for treating, alleviating, or preventing one or more symptoms associated with a pulmonary disease in dogs and cats. Non-limiting exemplary pulmonary diseases in dogs or cats include obstructive airway diseases (such as, but not limited to, Brachycephalic Obstructive Airway Syndrome (BOAS)), COPD, and allergic lung diseases such as asthma, bronchitis, or bronchial asthma.

The invention will be further described in the following examples, which do not limit the scope of the invention described in the claims.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1.-10. canceled

11. A method of treating a subject having a respiratory disorder or providing prophylaxis to a subject to prevent or reduce the severity of a developing respiratory disorder, comprising:
mechanically ventilating the subject with a breathing system; and
delivering a composition to the subject through the breathing system, wherein the composition comprises amniotic fluid, an amnion tissue preparation, or a combination thereof.

12. The method of claim 11, wherein the breathing system comprises a pressure-assisted breathing device.

13. The method of claim 12, wherein the pressure-assisted breathing device is a mechanical ventilator.

14. The method of claim 12, wherein the pressure-assisted breathing device is selected from the group consisting of an intensive care ventilator, a bubble ventilator, a continuous positive airway pressure system, a bi-level positive airway pressure system, an automatic positive airway pressure system, and an adaptive servo ventilation system.

15. The method of claim 11, wherein the breathing system further comprises a delivery device selected from a nebulizer, a metered dose inhaler, or a dry powder inhaler.

16. The method of claim 15, wherein the delivery device is operably connected to the breathing system to deliver the composition into the breathing system.

17. The method of claim 15, further comprising actuating the delivery device to deliver one or more doses of the composition into the breathing system and into the subject.

18. The method of claim 11, wherein the composition is delivered to the subject as a liquid, a solution aerosol, a suspension aerosol, or a nebulized aerosol.

19. The method of claim 11, wherein the composition is in the form of an aerosol or vapor in the breathing system.

20. The method of claim 11, wherein the composition is delivered in a particulate or droplet form having an average diameter of from about 0.1 microns to about 5 microns.

21. The method of claim 11, wherein the composition is delivered in particulate or droplet form having an average diameter of from about 1 micron to about 5 microns.

22. The method of claim 11, wherein the composition is delivered in particulate or droplet form having an average diameter of from about 2.5 microns to about 4.5 microns.

23. The method of claim 11, wherein the composition is delivered in particulate or droplet form having an average diameter of from about 3.5 microns to about 5 microns.

24. The method of claim 11, further comprising identifying the subject as having or at risk of developing the respiratory disorder.

25. The method of claim 11, further comprising identifying the respiratory disorder or one or more symptoms of the respiratory disorder.

26. The method of claim 11, wherein the respiratory disorder is selected from chronic obstructive pulmonary disease, asthma, acute asthma, chronic asthma, severe asthma, allergic asthma, bronchial asthma, intrinsic asthma, respiratory distress syndrome of the newborn, reversible respiratory disease, cystic fibrosis, bronchospasms, bronchitis, chronic bronchitis, bronchiectasis, alpha-1 antitrypsin emphysema, emphysema, associated cor pulmonale with pulmonary hypertension, right ventricular hypertrophy and right heart failure, pulmonary hypertension, interstitial lung disease, pulmonary fibrosis, pneumonia, interstitial pneumonia, a lung infection, idiopathic pulmonary fibrosis, cystic fibrosis, tuberculosis, severe acute respiratory syndrome, infection, pulmonary embolus, pulmonary arterial hypertension, pulmonary edema, pneumocystis pneumonia, SARS-CoV-2 infection, covid-19, acute respiratory distress syndrome, intensive care unit (ICU) syndrome, systemic inflammatory response syndrome (SIRS), sepsis, severe sepsis, septic shock, or multiple organ dysfunction syndrome (MODS), cystic fibrosis, sarcoidosis, and combinations thereof, and wherein the non-respiratory disorder is selected from an autoimmune disease, a spondyloarthropathy, an intestinal disease, diabetes, a skin disease, a non-respiratory infection, a pain disorder, intensive care unit (ICU) syndrome, systemic inflammatory response syndrome (SIRS), sepsis, severe sepsis, septic shock, or multiple organ dysfunction syndrome (MODS), cystic fibrosis, sarcoidosis, and combinations thereof.

27. The method of claim 11, wherein the amniotic fluid or the amnion tissue preparation lacks viable cells.

28. The method of claim 11, wherein the amniotic fluid or the amnion tissue preparation comprises viable cells.

29. The method of claim 11, wherein the composition consists essentially of amniotic fluid, an amnion tissue preparation, or a combination thereof.

30. A nebulizer comprising a composition comprising amniotic fluid, an amnion tissue preparation, or a combination thereof.

31. The nebulizer of claim 30, wherein the nebulizer is selected from a jet nebulizer, a soft mist nebulizer, an ultrasonic nebulizer, and a vibrating mesh nebulizer.

32. The nebulizer of claim 30, wherein the amniotic fluid or the amnion tissue preparation lacks viable cells.

33. The nebulizer of claim 30, wherein the amniotic fluid or the amnion tissue preparation comprises viable cells.

34. The nebulizer of claim 30, wherein the composition consists essentially of amniotic fluid, an amnion tissue preparation, or a combination thereof.

35.-74. (canceled)
